(19) United States
(12) Patent Application Publication
(10) Pub. No.: US 2025/0186995 A1
(43) Pub. Date: Jun. 12, 2025

(54) HIGH-THROUGHPUT SYSTEMS FOR INTRACELLULAR DELIVERY AND COMPONENTS AND USES THEREOF

(71) Applicant: Portal Biotechnologies, Inc., Arlington, MA (US)

(72) Inventors: Armon Reza SHAREI, Arlington, MA (US); Alec John BARCLAY, Winchester, MA (US); Sophia Madeleine HIRSCH, Boston, MA (US); Zhihui SONG, Philadelphia, PA (US); Andrew Francis LAROCQUE, Hamden, CT (US)

(21) Appl. No.: 18/975,945

(22) Filed: Dec. 10, 2024

Related U.S. Application Data
(60) Provisional application No. 63/608,757, filed on Dec. 11, 2023.

(51) Int. Cl. B01L 3/00; B01L 3/02; G01N 33/50

(52) U.S. Cl. CPC B01L 3/5025; B01L 3/0275; G01N 33/5008; B01L 2200/026; B01L 2200/04; B01L 2300/0681; B01L 2400/0487

(57) ABSTRACT

The present disclosure, in some aspects, is directed to high-throughput systems, and methods of use thereof, for intracellular delivery of one or more payloads to separate cell samples. For example, in certain aspects, provided is a high-throughput liquid handler configured for distribution of mechanically perturbed cells in formats suitable for high-throughput cellular assays. In certain other aspects, the present disclosure is directed to components of said high-throughput systems, e.g., liquid handlers and cartridges comprising a filter comprising a plurality of pores therethrough for perturbing cell membranes, and uses of said high-throughput systems.

### CROSS-REFERENCE TO RELATED APPLICATIONS

This application claims priority to and benefit of U.S. Provisional Patent Application 63/608,757, filed on Dec. 11, 2023, the contents of which are hereby incorporated herein by reference in their entirety and for all purposes.

### TECHNICAL FIELD

The present disclosure, in some aspects, is directed to high-throughput systems, and methods of use thereof, for intracellular delivery of one or more payloads to separate cell samples. For example, in certain aspects, provided is a high-throughput liquid handler configured for distribution of mechanically perturbed cells in formats suitable for high-throughput cellular assays. In certain other aspects, the present disclosure is directed to components of said high-throughput systems, e.g., liquid handlers and cartridges comprising a filter comprising a plurality of pores therethrough for perturbing cell membranes, and uses of said high-throughput systems.

### BACKGROUND

The ability to perform large-scale cellular analyses is broadly desirable across many disciplines, including for therapeutic drug candidate screens, gene editing assays, and pathway analyses. Involved in such analyses is the need to reliably and repeatably deliver payloads to cells, e.g., difficult to delivery small molecule drug candidates, proteins, and/or nucleic acids. Certain delivery techniques can have a negative impact on cells and result in assays that are not biologically meaningful. Thus, there remains a need for high-throughput systems, and components thereof, and methods for perturbing cells to enable delivery of payloads thereto and subsequent use of such cells in assays.

### BRIEF SUMMARY

The present application provides, in certain aspects, high-throughput systems for intracellular delivery, and components and uses of said systems. The high-throughput systems are configured for distribution of mechanically perturbed cells to a plurality of samples, wherein the samples can comprise one or more payloads prior to introduction of the perturbed cells such that the payloads are intracellularly delivered. The described systems, and methods of use, therefore enable large-format production of cells having desired payloads delivered thereto, and may find use in performing large-scale cellular assays, e.g., for drug screening. Such systems may comprise a single filter configured to process a large number of cells (such as at least about 1 million cells, including about 500 million cells), which the system then distributes to individual samples, such as to wells of a multi-well plate, e.g., a 96-, 384-, or 1536-well plate. The systems and methods taught herein can be performed in a high-throughput manner (e.g., a plate can have perturbed cell aliquots distributed to all wells in a manner of minutes) using various degrees of automation. Furthermore, the systems and methods taught herein can be used across a number of different cell lines including iPSCs, immune cells, and cancer cell lines such as HeLa, wherein the cells remain viable after processing and intracellular payload delivery.

Thus, in some aspects, provided herein is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples.

In some embodiments, the high-throughput liquid handler comprises a single filter comprising the plurality of pores for perturbing cell membranes of the plurality of cells. In some embodiments, the high-throughput liquid handler comprises a second filter comprising a plurality of pores therethrough for perturbing cell membranes of the plurality of cells, and the distributor is configured to dispense the liquid carrier comprising the mechanically perturbed cells from the second filter to two or more samples.

In some embodiments, the filter comprises at least about 1,000 pores. In some embodiments, the filter is configured to process at least about 1 million cells. In some embodiments, the filter is configured to process at least about 5 million cells in more than about 50 μL a liquid carrier. In some embodiments, the filter is configured to process at least about 5 million cells in about 30 minutes or less.

In some embodiments, the distributed, mechanically perturbed cells, when subjected to a condition to effect delivery of a payload to the two or more samples, exhibit about 20% or less change in cell viability as compared to the average.

In some embodiments, the high-throughput liquid handler is configured such that the residency time of the liquid carrier, as measured as the time between exiting the filter and exiting the one or more dispensing tips of the distributor to the sample, is about 2 minutes or less. In some embodiments, the residency time is about 30 seconds or less. In some embodiments, the residency time is about 0.01 seconds to about 5 seconds.

In other aspects, provided herein is a high-throughput liquid handler configured for distribution of mechanically perturbed cells, the high-throughput liquid handler comprising: a syringe comprising an outlet and a cell reservoir configured to hold a liquid carrier comprising a plurality of cells; a filter placed in fluidic communication with the cell reservoir of the syringe via the outlet, the filter comprising a plurality of pores therethrough for perturbing cell membranes of the plurality of cells; a fluid controller comprising a syringe pump configured to control the flow of the liquid carrier from the cell reservoir through the filter to produce the mechanically perturbed cells; a collection well situated to collect the liquid carrier comprising the mechanically perturbed cells from the filter; and a distributor comprising one or more dispensing tips for dispensing the liquid carrier comprising the mechanically perturbed cells from the collection well into two or more samples.

In other aspects, provided herein is a high-throughput liquid handler configured for distribution of mechanically perturbed cells, the high-throughput liquid handler comprising: a syringe comprising an outlet and a filter placed in the fluidic path of the outlet, the filter comprising a plurality of pores therethrough for perturbing cell membranes of the plurality of cells; a cell reservoir configured to hold a liquid carrier comprising a plurality of cells; a fluid controller comprising a syringe pump configured to control the flow of the liquid carrier through the filter such that the liquid carrier comprising the plurality of cells is aspirated from the cell reservoir through the filter and then dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

In other aspects, provided herein is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller comprising a solenoid configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples. In some embodiments, the reservoir is pressurized such that a force is applied to the liquid carrier therein in a manner that liquid carrier moves towards the filter. In some embodiments, the pressurization of the reservoir is a positive pressure. In some embodiments, the reservoir is pressurized with a gas. In some embodiments, the gas is air, CO₂, an inert gas, or any mixture thereof. In some embodiments, the reservoir is pressurized with a pump. In some embodiments, the pump is a peristaltic pump. In some embodiments, the filter, the fluid controller, and the distributor are configured as an end-effector comprising a solenoid positioned upstream and in fluidic communication with a filter positioned upstream and in fluidic communication with the one or more dispensing tips. In some embodiments, the end-effector is a removable and replaceable cartridge.

In other aspects, provided herein is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells is dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

In other aspects, provided herein is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells flows through the filter to produce mechanically perturbed cells; and a distributor comprising one or more dispensing tips for dispensing the liquid carrier comprising the mechanically perturbed cells from the syringe into two or more samples. In some embodiments, the distributor comprising the one or more dispensing tips is a gantry.

In other aspects, provided herein is a cartridge for use in a high-throughput liquid handlers described herein, wherein the cartridge comprises a filter comprising a plurality of pores therethrough for perturbing cell membranes of a plurality of cells, and a distributor comprising one or more dispensing tips for dispensing the mechanically perturbed cells into two or more samples.

In other aspects, provided herein is a cartridge for use in a high-throughput liquid handlers described herein, wherein the cartridge comprises a fluid controller comprising a solenoid configured to control the flow of a liquid carrier through a filter to produce mechanically perturbed cells, the filter comprising a plurality of pores therethrough for perturbing cell membranes of a plurality of cells, and a distributor comprising one or more dispensing tips for dispensing the mechanically perturbed cells into two or more samples.

In other aspects, provided herein is a method of performing a cellular assay, the method comprising preparing a plurality of cell-containing samples using a high-throughput liquid handler described herein, wherein the plurality of cell-containing samples comprises two or more samples separately having two or more populations of a cell comprising one or more different payloads delivered thereto; and performing the cellular assay on plurality of cell-containing samples. In some embodiments, the method comprises preparing at least about 100 cell-containing samples. In some embodiments, the plurality of cell-containing samples is prepared in 30 minutes or less.

In other aspects, provided herein is a method of performing a cellular assay, the method comprising preparing a plurality of cell-containing samples comprising: passing a plurality of cells through a filter comprising a plurality of pores therethrough for perturbing cell membranes of the plurality of cells; distributing the mechanically perturbed cells to the plurality of cell-containing samples, wherein the plurality of cell-containing samples comprises two or more samples separately having two or more populations of a cell comprising one or more different payloads delivered thereto; and performing the cellular assay on plurality of cell-containing samples.

### BRIEF DESCRIPTION OF THE DRAWINGS

FIG. 1 shows a schematic of generalized aspects of the systems described herein.

FIG. 2 shows a schematic of generalized aspects of the systems described herein.

FIGS. 3A and 3B show diagrams of an exemplary end-effector 400 comprising a solenoid 402 positioned upstream and in fluidic communication with a filter in a filter housing 404, the filter positioned upstream and in fluidic communication with a dispensing tip 406.

FIGS. 4A and 4B show plots of flow cytometry results demonstrating the intracellular delivery of dextran to iPSCs across various residency times.

FIGS. 5A and 5B show plots of flow cytometry results demonstrating the intracellular delivery of dextran to iPSCs across various residency times. FIG. 5C shows a plot of cell viability of iPSCs following intracellular dextran delivery across various residency times.

FIGS. 6A and 6B show plots of flow cytometry results demonstrating the intracellular delivery of dextran to HeLa cells across various residency times and filter pore sizes. FIG. 6C shows a plot of cell viability of HeLa cells following intracellular dextran delivery across various residency times and filter pore sizes.

FIGS. 7A and 7B show plots of flow cytometry results demonstrating the intracellular delivery of dextran to activated T cells across various residency times. FIG. 7C shows a plot of cell viability of activated T cells following intracellular dextran delivery across various residency times.

FIGS. 8A and 8B show plots of flow cytometry results demonstrating the intracellular delivery of dextran to activated T cells across various residency times. FIG. 8C shows a plot of cell viability of activated T cells following intracellular dextran delivery across various residency times.

FIGS. 9A and 9B show plots of flow cytometry results demonstrating the intracellular delivery of dextran to HeLa cells across using various pressure settings in a high-throughput system. FIG. 9C shows a plot of cell viability of HeLa cells following intracellular dextran delivery. FIG. 9D illustrates the configuration of a filter, solenoid, and dispenser of the demonstrated high-throughput system.

FIG. 10A illustrates a cross-sectional view of example cartridge for cell mechanoporation. FIG. 10B illustrates the example cartridge for cell mechanoporation. FIG. 10C illustrates an exploded view of the example cartridge for cell mechanoporation showing the cartridge holding a polymer filter and mesh support. FIG. 10D illustrates an exploded view of the exemplary cartridge for cell mechanoporation, the cartridge holding a silicon filter and sealing component, in accordance with some embodiments.

FIGS. 11A and 11B show plots of flow cytometry results of dextran delivery to live PBMCs (FIG. 11A) and cell viability following mechanoporation (FIG. 11B).

FIG. 12A shows a plot of flow cytometry results of dextran delivery to live iPSCs for a high-throughput system (HTS) described herein and a control research scale system. FIG. 12B shows a plot of flow cytometry results of cell viability following mechanoporation for a high-throughput system (HTS) described herein and a control research scale system. FIG. 12C shows a plot of volumetric flow rates for various pressures used in operating a high-throughput system described herein. FIG. 12D shows a plot of flow cytometry results of retention of dextran delivered to cells using a high-throughput system (HTS) described herein and a control research scale system.

FIGS. 13A and 13B show plots of flow cytometry results demonstrating the intracellular delivery of a macrocyclic peptide to cell wherein the macrocyclic peptide is admixed with already mechanoporated cells. FIG. 13C shows a plot of cell viability following mechanoporation and delivery of the macrocyclic peptide.

### DETAILED DESCRIPTION

Provided in the present application, in certain aspects, are high-throughput systems, and methods and components thereof, for producing mechanically perturbed cells using a filter, the filter comprising a plurality of pores therethrough, for the intracellular delivery of payloads. The present application is based, at least in part, on the inventors' findings demonstrating that a single filter comprising a plurality of pores therethrough for perturbing cell membranes of a plurality of cells can be used to can be used to generate a plurality of samples comprising perturbed cells and that these perturbed cells can then be exposed to one or more payloads for intracellular delivery. In some embodiments, thus provided is a high-throughput system comprising a single filter configured to produce two or more samples of perturbed cells. In some embodiments, provided is a high-throughput system comprising more than one filter, wherein each filter is configured to produce two or more samples of perturbed cells. As described herein, efficient intracellular delivery of a payload can be achieved using the systems, and components thereof, described herein when there is a time delay between a cell exiting a filter and being exposed to a payload (referred to herein as residency time), as compared to mechanically perturbing a cell in the presence of a payload. Such unexpected findings regarding the use of filters and residency time demonstrated herein enabled the development of high-throughput systems and methods for intracellular delivery on a scale not known to be possible at the time of filing the instant application. For example, as provided herein, large-format sample plates (such as 96-well plates, 384-well plates, or 1536-well plates) can be pre-loaded with one or more payloads, and then the mechanically perturbed cells can be generated and added to each well to accomplish intracellular delivery. Such systems and methods enable the formation of any number of samples of cells having different payloads delivered thereto (and/or replicates), and can be performed to produce a large number of separate cell samples in a high-throughput manner, e.g., production and distribution of perturbed cells to all wells of a 1536-well plate within 10 minutes, e.g., 5 minutes or less. In some embodiments, the systems are configured such that a single filter can process at least about 1 million cells, such as at least about 100 million cells. Moreover, the high-throughput systems described herein can be used to deliver payloads to different cell lines, such as iPSCs, immune cells, and cancer cell lines such as HeLa, wherein the cells remain viable after processing and intracellular payload delivery. The inventions described herein provide a solution to many existing challenging currently faced in the study of cells, and provide new approaches for performing, e.g., drug screening, cell-based therapeutics, and genetics. In some embodiments, the high-throughput systems, and methods of use thereof, are highly automated and require little human supervision to produce cell samples. In some embodiments, certain steps may be performed manually, such as pipetting or generating a population of perturbed cells prior to distribution.

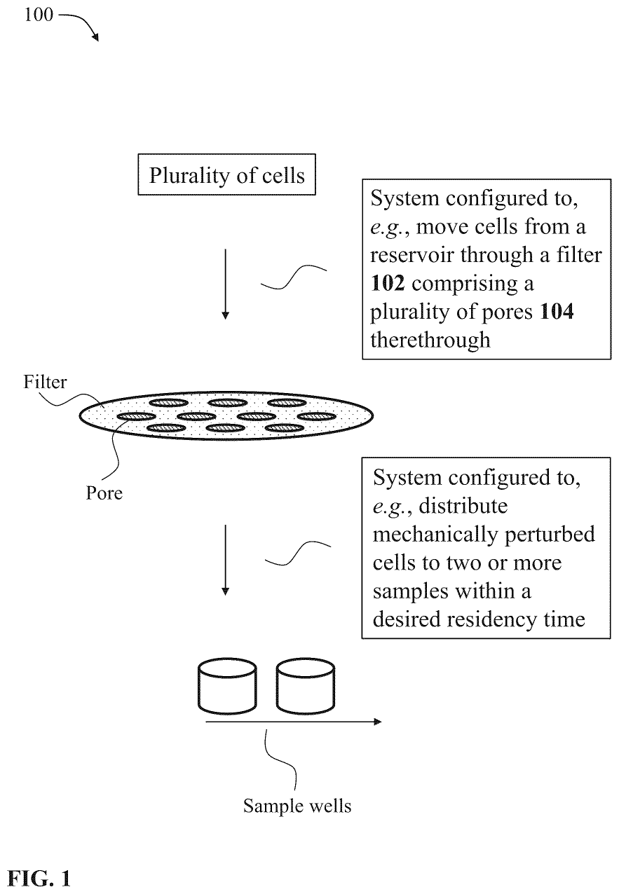

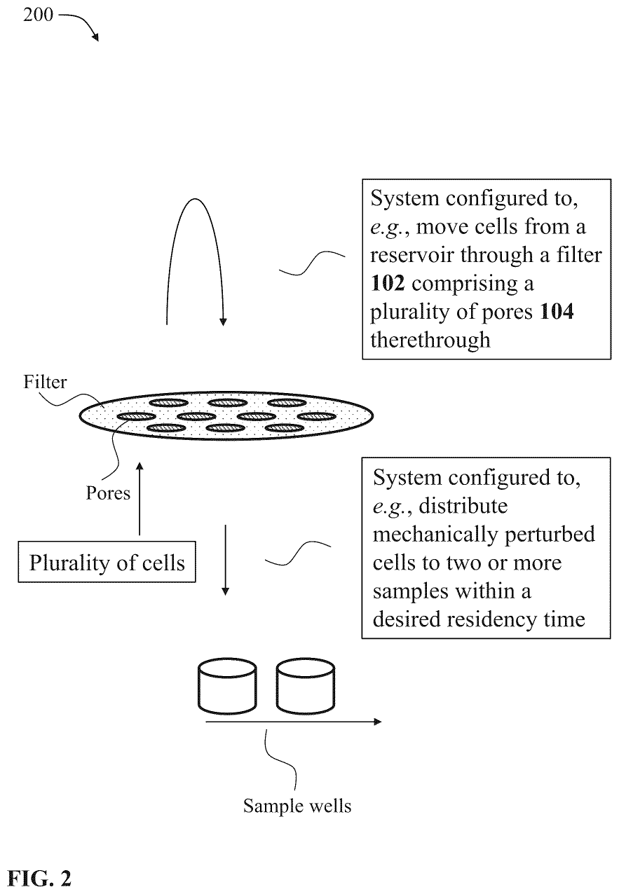

For purposes of facilitating the understanding of the description provided herein, FIGS. 1 and 2 are provided to show generalized aspects of certain workflows of the systems and methods described herein. As illustrated in FIG. 1, in some embodiments, the system 100 is configured to move a plurality of cells through a filter comprising a plurality of pores therethrough for perturbing cell membranes of the plurality of cells. In some embodiments, the plurality of cells is contained in a cell reservoir (e.g., a pressurized container or the barrel of a syringe). The plurality of cells can be passed through the filter and then distributed to two or more samples. A fluid controller can be used to control the flow of a liquid carrier comprising the plurality of cells through the filter. In some embodiments, the fluid controller comprises a solenoid, wherein the liquid carrier is held in a cell reservoir and the solenoid controls the flow of liquid carrier through the filter. In some embodiments, the fluid controller comprises a syringe pump (or equivalent), wherein the liquid carrier is held in a barrel of a syringe and can be pushed through the filter. In some embodiments, the mechanically perturbed cells are distributed using one or more dispensing tips (e.g., an end-effector or a pipette tip). The one or more dispensing tips may be in fluidic communication with the filter such that mechanically perturbed cells are more directly dispensed to form samples, such as into separate wells of a multi-well plate. In other embodiments, the one or more dispensing tips are configured to dispense (and aspirate as necessary) mechanically perturbed cells sourced from a central source, e.g., a trough holding processed liquid carrier comprising the mechanically perturbed cells. In certain other embodiments described herein, the plurality of cells may be aspirated through a filter prior to being flowed back through the filter, and thus may travel through the filter twice (concept is illustrated in FIG. 2). Such embodiments are useful for certain systems configurations taught herein. As described in detail herein, in some embodiments the systems are configured to, e.g., operate with a single filter which can process a large number of cells (e.g., at least about 5 million cells to about 500 million cells) in a short about of time (e.g., less than 30 minutes). The scale and speed of the systems described herein enable a diverse array of high-throughput cellular assays that are highly reproducible. The concepts of the system can be similarly applied to the methods described herein. In some embodiments, certain steps are performed with manual intervention (e.g., pipetting or generating a population of perturbed cells prior to distribution).

Thus, provided herein, in some aspects, is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples.

Provided herein, in other aspects, is a high-throughput liquid handler configured for distribution of mechanically perturbed cells, the high-throughput liquid handler comprising: a syringe comprising an outlet and a cell reservoir configured to hold a liquid carrier comprising a plurality of cells; a filter placed in fluidic communication with the cell reservoir of the syringe via the outlet, the filter comprising a plurality of pores therethrough for perturbing cell membranes of the plurality of cells; a fluid controller comprising a syringe pump configured to control the flow of the liquid carrier from the cell reservoir through the filter to produce the mechanically perturbed cells; a collection well situated to collect the liquid carrier comprising the mechanically perturbed cells from the filter; and a distributor comprising one or more dispensing tips for dispensing the liquid carrier comprising the mechanically perturbed cells from the collection well into two or more samples.

Provided herein, in other aspects, is a high-throughput liquid handler configured for distribution of mechanically perturbed cells, the high-throughput liquid handler comprising: a syringe comprising an outlet and a filter placed in the fluidic path of the outlet, the filter comprising a plurality of pores therethrough for perturbing cell membranes of the plurality of cells; a cell reservoir configured to hold a liquid carrier comprising a plurality of cells; a fluid controller comprising a syringe pump configured to control the flow of the liquid carrier through the filter such that the liquid carrier comprising the plurality of cells is aspirated from the cell reservoir through the filter and then dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

Provided herein, in other aspects, is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller comprising a solenoid configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples. In some embodiments, the distributor comprising the one or more dispensing tips is a gantry.

Provided herein, in other aspects, is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells is dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

Provided herein, in other aspects, is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells flows through the filter to produce mechanically perturbed cells; and a distributor comprising one or more dispensing tips for dispensing the liquid carrier comprising the mechanically perturbed cells from the syringe into two or more samples.

Provided herein, in other aspects, is a cartridge for use in a high-throughput liquid handlers described herein, wherein the cartridge comprises a filter comprising a plurality of pores therethrough for perturbing cell membranes of a plurality of cells, and a distributor comprising one or more dispensing tips for dispensing the mechanically perturbed cells into two or more samples.

Provided herein, in other aspects, is a cartridge for use in a high-throughput liquid handlers described herein, wherein the cartridge comprises a fluid controller comprising a solenoid configured to control the flow of a liquid carrier through a filter to produce mechanically perturbed cells, the filter comprising a plurality of pores therethrough for perturbing cell membranes of a plurality of cells, and a distributor comprising one or more dispensing tips for dispensing the mechanically perturbed cells into two or more samples.

Provided herein, in other aspects, is a method of performing a cellular assay, the method comprising preparing a plurality of cell-containing samples using a high-throughput liquid handler described herein, wherein the plurality of cell-containing samples comprises two or more samples separately having two or more populations of a cell comprising one or more different payloads delivered thereto; and performing the cellular assay on plurality of cell-containing samples.

Provided herein, in other aspects, is a method of performing a cellular assay, the method comprising preparing a plurality of cell-containing samples comprising: passing a plurality of cells through a filter comprising a plurality of pores therethrough for perturbing cell membranes of the plurality of cells; distributing the mechanically perturbed cells to the plurality of cell-containing samples, wherein the plurality of cell-containing samples comprises two or more samples separately having two or more populations of a cell comprising one or more different payloads delivered thereto; and performing the cellular assay on plurality of cell-containing samples.

The disclosures of all publications, patents, patent applications and published patent applications referred to herein are hereby incorporated herein by reference in their entirety.

### I. Definitions

As used herein, the term “individual” refers to a mammal and includes, but is not limited to, human, bovine, horse, feline, canine, rodent, rat, mouse, dog, or primate (such as a non-human primate). In some embodiments, the individual is a human individual.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### II. High-Throughput Liquid Handler Systems

In certain aspects, provided herein are high-throughput liquid handler systems for distribution of mechanically perturbed cells. In some embodiments, the high-throughput systems described herein are configured to produce at least 10 samples of perturbed cells (such as samples between 5 μL and 200 μL containing a wide range of cells, e.g., at least about 1 million cells per sample), such as at least 10 samples within 10 minutes or less, such as any of 9 minutes or less, 8 minutes or less, 7 minutes or less, 6 minutes or less, 5 minutes or less, 4 minutes or less, 3 minutes or less, 2 minutes or less, or 1 minute or less. In some embodiments, the high-throughput system can produce significantly larger numbers of samples at the pace described above, such as useful for producing samples for larger formats, e.g., 96-well plates or above, including 384-well plates and 1536 well-plates. In some embodiments, the high-throughput system is configured to produce samples for one plate (such as for a 96-, 384-, or 1536-well plate) with a single filter comprising a plurality of pores therethrough for perturbing cell membranes of cells. As described in more detail below, the systems taught herein comprise, e.g., a filter comprising a plurality of pores therethrough for perturbing cell membranes of cells, a fluid controller configured to control the flow of a liquid carrier comprising cells from a cell reservoir through a filter to produce mechanically perturbed cells, a distributor comprising one or more dispensing tips for dispensing a liquid carrier comprising mechanically perturbed cells into two or more samples, and a cell reservoir to hold a liquid carrier comprising cells.

In some embodiments, provided is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells, a fluid controller configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples. In some embodiments, the high-throughput liquid handler comprises a single filter comprising the plurality of pores for perturbing cell membranes of the plurality of cells. In some embodiments, the high-throughput liquid handler comprises a second filter comprising a plurality of pores therethrough for perturbing cell membranes of the plurality of cells, and the distributor is configured to dispense the liquid carrier comprising the mechanically perturbed cells from the second filter to two or more samples.

As further elaborated herein, in some embodiments, the filter comprises at least about 1,000 pores, such as at least about any of 5,000 pores, 10,000 pores, 20,000 pores, 30,000 pores, 40,000 pores, 50,000 pores, 60,000 pores, 70,000 pores, 80,000 pores, 90,000 pores, or 100,000 pores. In some embodiments, the filter is configured to process at least about 1 million cells (such as between about 1 million cells to about 500 million cells). In some embodiments, the filter is configured to process at least about 1 million cells (such as between about 1 million cells to about 500 million cells) in more than about 50 μL a liquid carrier. In some embodiments, the filter is configured to process at least about 1 million cells (such as between about 1 million cells to about 500 million cells) in about 30 minutes or less, such as about any of 25 minutes or less, 20 minutes or less, 19 minutes or less, 18 minutes or less, 17 minutes or less, 16 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, 5 minutes or less, 3 minutes or less, 2 minutes or less, or 1 minute or less. In some embodiments, the distributed, mechanically perturbed cells, when subjected to a condition to effect delivery of a payload to the two or more samples (such as introducing a payload after a desired residency time), exhibit about 20% or less change in cell viability as compared to the average (such as based on replicates), a starting sample and an ending sample after processing of at least about 5 million cells, and/or as compared to a control not subjected to mechanical portion.

In some embodiments, the high-throughput liquid handler is configured to dispense a sample comprising mechanically perturbed cells to 96 individual wells of a multi-welled plate within about 30 seconds. In some embodiments, the high-throughput liquid handler is configured to dispense a sample comprising mechanically perturbed cells to 384 individual wells of a multi-welled plate within about 30 seconds. In some embodiments, the high-throughput liquid handler is configured to dispense a sample comprising mechanically perturbed cells to 1536 individual wells of a multi-welled plate within about 30 seconds.

The high-throughput systems disclosed herein may be described using terms for components, such as a filter, a fluid controller, and a distributor. It is to be understood that use of such terms, including their separate use, is not intended to mean that such components must only be separatable components, e.g., in some embodiments a fluid controller is integrated with a dispenser as one physical component and the boundary between them is arbitrarily defined. In some embodiments, the components are separate or separatable. The high-throughput systems disclosed herein may comprise a variety of different combinations of component features described herein. In some instances, such high-throughput systems and component features are described in a modular fashion. The modular discussion of such component features does not limit the scope of the inventions provided herein and one of ordinary skill in the art will readily appreciate how certain features from the sections below can be combined to provide the high-throughput liquid handlers taught herein.

### A. Residency Times

As discussed herein, the systems utilize a time between passing cells through a filter for perturbing cell membranes and subsequent introduction to payload to distribute the mechanically perturbed cells in a format suitable for, e.g., high-throughput screening.

In some embodiments, the high-throughput liquid handler is configured such that the residency time of a liquid carrier, as measured as the time between exiting the filter and exiting the one or more dispensing tips of the distributor to the sample, is about 2 minutes or less, such as about any of 110 seconds or less, 100 seconds or less, 90 seconds or less, 80 seconds or less, 70 seconds or less, 60 seconds or less, 50 seconds or less, 40 seconds or less, 30 seconds or less, 20 seconds or less, 15 seconds or less, 10 seconds or less, 5 seconds or less, 4 seconds or less, 3 seconds or less, 2 seconds or less, or 1 second or less. In some embodiments, the residency time is about 30 seconds or less. In some embodiments, the residency time is about 0.01 seconds to about 5 seconds. As described in other aspect of the instant application, features of the system and components thereof can contribute to residency time, such as the volume of a dispensing tip and speed of dispensing. In some embodiments, the structure of one or more components of the high-throughput system is designed to satisfy a residency time metric, e.g., the dead volume after the filter, such as of a dispensing tip, is less than about 5 mL, such as less than about any of 4.5 mL, 4 mL, 3.5 mL, 3 mL, 2.5 mL, 2 mL, 1.5 mL, 1 mL, 0.9 mL, 0.8 mL, 0.7 mL, 0.6 mL, 0.5 mL, 0.4 mL, 0.3 mL, 0.2 mL, or 0.1 mL. From such description, one of ordinary skill in the art will readily appreciate the systems and components taught herein to perform distribution of mechanically perturbed cells within a desired residency time such that intracellular payload delivery occurs.

### B. Filters Comprising a Plurality of Pores Therethrough

The filters provided herein comprise a plurality of pores therethrough for perturbing cell membranes cells such that intracellular delivery can occur when the mechanically perturbed cell is in the presence of a payload within a set amount of time.

The filter may comprise a silicon filter or a polymer filter. Unless explicitly stated otherwise, it is to be understood that features described herein with respect to the silicon filter are understood to be applicable to the polymer filter, and vice versa.

The silicon filter may comprise a filtering surface and a support structure disposed on a side of the filtering surface. The support structure may cover at least a portion of the filtering surface. The filter may comprise a plurality of pores extending through the filtering surface, the pores configured to perturb cell membranes as a cell mixture passes through the plurality of pores.

The silicon filter may comprise silicon, silicon oxide (e.g., silica), silicon nitride, and/or silicon carbide. For example, the filtering surface may comprise one or more of the aforementioned silicon materials. Likewise, the support structure may comprise one or more of the aforementioned silicon materials. The filtering surface and the support structure may comprise the same or a different silicon material. The silicon filter may be fabricated from a silicon wafer that is doped. For example, the silicon wafer used to fabricate the silicon filter may be doped with boron, gallium, or phosphorous.

The polymer filter may comprise a plurality of pores etched through a polymer substrate, the pores extending through the filtering surface of the filter. Although not explicitly illustrated, it is to be understood that the polymer filter may comprise a support structure, such as a mesh, disposed on a side of the filtering surface. The mesh may be disposed proximate to the filtering surface, or the mesh may be attached to the polymer filtering surface to support the polymer filtering surface.

The polymer filter may comprise polycarbonate, polyester (PET), polyethersulfone, polyacrylonitrile (PAN), polypropylene, PVDF, and/or polytetrafluorethylene. For example, a filtering surface of the polymer filter may comprise one or more of the aforementioned polymer materials. In some embodiments, the support structure (e.g., mesh) may comprise one or more of the aforementioned materials. In some embodiments, the support structure and filtering surface of the polymer filter may comprise the same or a different polymer material.

The filter may be coated. One or more sides of the filter may be coated with a coating. For example, the filter may comprise a coating including one or more materials such as gold, silver, platinum, Teflon, polyvinylpyrrolidone, an adhesive coating, surfactants, one or more proteins, adhesion molecules, antibodies, anticoagulants, factors that modulate cellular function, nucleic acids, lipids, carbohydrates, and/or transmembrane proteins. In some embodiments, the filter may not comprise a coating.

The filter may comprise several hundred, thousand, or tens of hundreds (or thousand) pores for perturbing cell membranes. For example, the filter may comprise between about 500-400,000 pores, 500-100,000 pores, 10,000-400,000 pores, 10,000-100,000 pores, 50,000-400,000 pores, 50,000-100,000 pores, or 100,000-400,000 pores. In some embodiments, the filter may comprise at least about 500, 1,000, 5,000, 10,000, 50,000, 75,000, 100,000, 125,000, 150,000, 175,000, 200,000, 225,000, 250,000, 275,000, 300,000, 325,000, 350,000, or 375,000 pores. In some embodiments, the filter may comprise no more than 1,000, 5,000, 10,000, 50,000, 75,000, 100,000, 125,000, 150,000, 175,000, 200,000, 225,000, 250,000, 275,000, 300,000, 325,000, 350,000, 375,000, or 400,000 pores.

In some embodiments, the plurality of pores extending through the filtering surface may comprise uniformly-sized pores or varying pore sizes throughout the surface. Different sized pores may lend to varying efficacies of perturbing cell membranes of the different types of cells that may be passed through the plurality of pores. The plurality of pores may comprise one or more cross-sectional shapes including but not limited to circular, rectangular (e.g., square), elliptical, triangular, or another polygonal shape. Because the filtering surface exhibits a thickness (described in greater detail below), it is understood that the plurality of pores can exhibit a three-dimensional shape (e.g., a prism) extending through the filtering surface. For example, in the instance a pore comprises a circular cross-section with a uniform diameter along the thickness of the filtering surface, the 3-dimensional shape of the pore may be a cylinder. In the instance the pore comprises a triangular shape, the 3D shape of the pore may be a triangular prism. In the instance the width of the pore varies (e.g., increases or decreases) along the thickness of the filtering surface, the 3D shape of the pore can be conical.

In some embodiments, a width of each pore of the plurality of pores may be between 2 μm and 20 μm. The width of the pore may be dependent on the type of cell(s) in the cell mixture passed through the plurality of pores. For example, the width may be between about 2-15 μm, 2-10 μm, 2-7 μm, 8-15 μm, 8-10 μm, or about 11-15 μm. In some embodiments, the pore width may be greater than or equal to about 2, 4, 6, 8, 10, 12, 14, 15, 16, or 18 μm, such as measured as an average pore width. In some embodiments, the pore width may be less than or equal to about 4, 6, 8, 10, 12, 14, 15, 16, 18, or 20 μm. In some embodiments, each pore of the plurality of pores may comprise a circular cross-section, and the width of the pore may be a diameter of the pore.

As mentioned above, the width of a given pore in the filter may be dependent on the cell type being passed through the plurality of pores. For example, the width of each of the pores may be smaller than the diameter of the cells in the mixture passing through the filter, such that forcing the cell through the pore under pressure causes a perturbation in the membrane of the cell as the cell is constricted by the pore. For example, the pore width may be between 10% and 99% of the diameter of the cells in a cell suspension, such as about 10%, 20%, 30%, 40%, 50%, 60%, 70%, 80%, 90%, 95%, 98%, or 99% of the diameter of the cells. It is to be understood that reference to a diameter of a cell is intended as the diameter of the cell in the cell mixture prior to being passed through the filter, e.g., as the cell approaches the filter, unless otherwise specified.

In some embodiments, a pitch between each pore of the plurality of pores may be between 0.5:1 and 100:1 relative to the width of the pore. The pitch of the pore may be selected to allow the cell mixture to flow through the filter without backing up, while also maintaining the structure and ability of the filter to withstand various flow rate and pressure conditions. In some embodiments, the pitch between pores may be between about 0.5:1 and 5:1, 0.5:1 and 10:1, 0.5:1 and 25:1, or 0.5:1 and 50:1 relative to the width of the pore. In some embodiments, the pitch between pores may be greater than or equal to about 0.5:1, 0.75:1, 1:1, 2:1, 3:1, 4:1, 5:1, 10:1, 15:1, 20:1, 25:1, 50:1, 75:1, or 100:1. In some embodiments, the pitch between pores may be less than or equal to about 0.5:1, 0.75:1, 1:1, 2:1, 3:1, 4:1, 5:1, 10:1, 15:1, 20:1, 25:1, 50:1, 75:1, or 100:1. In non-limiting example, a pore width between 2-7 μm may have a 4:1 pitch, meaning a 2-μm pore may be spaced 8 μm apart from another 2-μm pore. The pores may be randomly distributed on the polymer filtering surface.

The plurality of pores may cover between about 0.1% and about 60% of the total surface area of the filter. In some embodiments, the plurality of pores may comprise between about 1.0×10¹ to about 1.0×10¹⁰ pores per square millimeter of total surface area of the filter. The porous surface may in some embodiments comprise between about 1.0×10¹ to about 1.0×10¹⁵ pores/mm² total surface area of the filter.

In some embodiments, each pore of the plurality of pores may extend linearly through the filtering surface. For example, the pores may extend through the filter in a direction collinear with the thickness dimension of the filter. In some embodiments, the linear extension of the pores through the filtering surface may be collinear with the direction of flow of the cell mixture through the pores.

In some embodiments, a thickness of the filtering surface may be between 0.1 μm and 100 μm. As mentioned above, the thickness of the filtering surface may be defined by the axis along which the cell mixture travels as it passes through the filter. The thickness of filtering surface may consider the pore length necessary to perturb the cell membrane while minimizing clogging within the pores. Also, as described herein with respect to other characteristics of the filter, the thickness of filtering surface may be selected to withstand breakage during use. In some embodiments, the thickness of the filtering surface may be between about 0.1-10 μm, 1-100 μm, 1-10 μm, or 10-100 μm. In some embodiments, the thickness of the filtering surface 522 may be greater than or equal to about 0.1, 0.5, 1, 2, 5, 10, 15, 20, 25, 50, 75, or 100 μm. In some embodiments, the thickness of the filtering surface 522 may be less than or equal to about 0.1, 0.5, 1, 2, 5, 10, 15, 20, 25, 50, 75, or 100 μm.

The filter may comprise a circular, triangular, rectangular, elliptical, or other polygonal shape. For example, the filtering surface of the filter can include a rectangular (e.g., square) shape. In some embodiments, the filter can be described by a width, wherein the width extends from a first side to a second side of the filtering surface opposite the first side. In some embodiments, the width of the filtering surface may be between 1 mm and 10 cm. For example, the width of the filtering surface may be between about 1 mm-5 cm, 1 mm-1 cm, 5 mm-10 cm, 5 mm-1 cm, 1 cm-10 cm, 1 cm-5 cm, or 5 cm-10 cm. In some embodiments, the width of the filtering surface may be greater than or equal to 1 mm, 2 mm, 5 mm, 8 mm, 1 cm, 2 cm, 5 cm, 8 cm, or 10 cm. In some embodiments, the width of the filtering surface may be less than or equal to 1 mm, 2 mm, 5 mm, 8 mm, 1 cm, 2 cm, 5 cm, 8 cm, or 10 cm.

In some embodiments, the filter can be described by a length, wherein the length of the filtering surface may be substantially the same as the width of the filtering surface. In some embodiments, the length of the filtering surface may be greater than or less than the width of the filtering surface. Any of the above example ranges of or values for the width of the filtering surface are understood to be applicable to the length of the filtering surface. In some embodiments, the filtering surface can include a circular shape. In this instance, the width may be understood to be the diameter of the filtering surface.

The shape and size of the filter may be selected to correspond with upstream fluid inlets and/or downstream fluid outlets. For example, the width of the filtering surface may be greater than or equal to that of the fluid inlet and/or the fluid outlet to limit dead volume between the fluid pathways and the filter. In some embodiments, the shape of the filtering surface may correspond to that of the fluid inlet and/or the fluid outlet. For example, in the instance the fluid inlet and/or the fluid outlet comprises a circular cross-section, the filtering surface may comprise a circular shape.

The filtering surface is described with reference to a single filter. It is to be understood that a system may in some embodiments comprise a plurality of filters. For example, the filters may be arranged in parallel to create an array of filters that together encompass a larger filtering surface. Any of the dimensions provided above with respect to the filtering surface are understood to be applicable to this filtering surface composed of a plurality of filters. For example, a plurality of filters, each filter comprising a width of 1 mm, may be arranged in array to create a filtering surface comprising a width on the magnitude of 1-10 cm.

As described herein, a silicon filter may comprise a support structure disposed on a side of the filtering surface. In some embodiments, a thickness of the support structure may be between 20 μm and 1 mm. Similar to the filtering surface, the thickness of the support structure may be defined by the axis along which the cell mixture travels as it passes through the filter. The thickness of support structure may be selected such that the support structure enables the silicon filter to withstand breakage during use, but also does not impede on the flow of the cell mixture through the pores of the filtering surface. In some embodiments, the thickness of the support structure may be between about 20 μm-100 μm, 50 μm-1 mm, 50 μm-100 μm, 0.1-1 mm, 0.1-0.5 mm, or 0.5-1 mm. In some embodiments, the thickness of the support structure may be greater than or equal to about 20 μm, 50 μm, 80 μm, 0.1 mm, 0.2 mm, 0.5 mm, 0.8 mm, or 1 mm. In some embodiments, the thickness of the support structure may be less than or equal to about 20 μm, 50 μm, 80 μm, 0.1 mm, 0.2 mm, 0.5 mm, 0.8 mm, or 1 mm.

In some embodiments, the support structure may cover at least a portion of the filtering surface, such as about 1% of the filtering surface. The support structure can cover at least a portion of the filtering surface to support the filtering surface and prevent it from breaking in various flow rate and pressure conditions induced on the filter. However, the support structure has to allow the cell mixture to pass through the filtering surface without flow backing up and/or the filtering clogging. Thus, the support structure can cover a portion of the filtering surface. In some embodiments, the support structure may cover between about 0.1-30%, 0.1-10%, 0.1-1%, 1-30%, 1-10%, or 1-5% of the silicon filtering surface. In some embodiments, the support structure may cover greater than or equal to about 0.1%, 0.2%, 0.5%, 0.8%, 1%, 1.5%, 2%, 2.5%, 3%, 4%, 5%, 6%, 7%, 8%, 9%, 10%, 12%, 15%, 18%, 20%, 22%, 25%, 28%, or 30% of the filtering surface. In some embodiments, the support structure may cover less than or equal to about 0.1%, 0.2%, 0.5%, 0.8%, 1%, 1.5%, 2%, 2.5%, 3%, 4%, 5%, 6%, 7%, 8%, 9%, 10%, 12%, 15%, 18%, 20%, 22%, 25%, 28%, or 30% of the filtering surface. In some embodiments, the support structure may cover greater than 30% of the filtering surface, such as about 35%, 40%, 45%, or 50% of the filtering surface.

The support structure may comprise one or more supporting members disposed on the filtering surface and extending in one or more directions.

The one or more supporting members can create two or more filtering windows in the filtering surface. The support structure can comprise a plurality of supporting members disposed on the filtering surface, and the plurality of supporting members together can form at least one cross shape that separates a plurality of filtering windows in the filtering surface. For example, the support structure can be composed of 2, 3, 4, 5, 6, 7, 8, 9, 10, or more or more supporting members. The supporting members can form 1, 2, 3, 4, 5, 6, 7, 8, 9, 10 or more cross-shapes on the filtering surface. The supporting members can create 2, 3, 4, 5, 6, 7, 8, 9, 10, 11, 12, 13, 14, 15 or more filtering windows in the filtering surface.

The support structure is not limited to the cross-shape(s) described herein. For example, the supporting member creating the central filtering window may comprise a circular, ovular, triangular, rectangular (e.g., square), or other polygonal shape. In some embodiments, a support structure comprising curved supporting members (e.g., circular supporting members, like the central filtering window) may improve the filter's ability to withstand pressure and flow conditions induced during cell mechanoporation, at least in comparison to support structures including one or more sharp corners or edges. One or more supporting members may extend from the central supporting member toward the perimeter of the filtering surface. For example, 1, 2, 3, 4, 5, 6, 7, 8 or more supporting members may extend from the central supporting member, thereby creating one or more filtering windows adjacent to the central filtering window.

In some embodiments, the support structure may comprise one or more supporting members disposed independently as stripes on the filtering surface. A, including each, supporting member of the support structure may extend from a side of the filtering surface toward the side of the filtering surface opposite the aforementioned side. A given supporting member of the support structure may extend substantially all the way from one side to the or may extend only part of the way. For example, a width of the support structure may be about 10%, 20%, 30%, 40%, 50%, 60%, 70%, 80%, 90%, 95%, or 99% of the width of the filtering surface. The supporting members may be disposed in an alternating fashion, or alternatively may all extend from one side of the filtering surface or the other.

The filter may comprise a border surrounding the filtering surface. The border may be a non-porous material—in other words, the border may not comprise pores therethrough. This border may be used to retain the filter within a cartridge without obstructing the filtering surface. In some embodiments, the filter (including the border) may comprise a circular, triangular, rectangular, elliptical, or other polygonal shape. In some embodiments, the shape of the filter may mimic that of the filtering surface. In some embodiments, the shape of the filter and the filtering surface may be different (e.g., the filter including the border can comprise rectangular shape, whereas the filtering surface can comprise a circular shape). The size of the filter is not intended to be limited to the disclosure provided herein. In a few non-limiting examples, the filter may be 2×2 mm, 5×5 mm, 10×10 mm, or any dimensions therebetween.

In some embodiments, the filter may comprise one or more oxide layers. For example, an oxide layer may be disposed on the filtering surface between the filtering surface and the support structure. The filter may comprise an oxide layer to ease manufacturing each of filtering surface and the support structure from a silicon wafer. For example, fabrication of a silicon filter may comprise etching a silicon filtering layer and a silicon support layer of a silicon wafer, the silicon layers separated by an oxide layer. With the oxide layer between the filtering layer and the support layer, each of the filtering surface and the support structure can be reliably etched without etching the other side.

In some embodiments, all of the liquid carrier may be passed through a filter in about 30 minutes or less. In some embodiments, all of the liquid carrier may be passed through a filter in no more than about 30 minutes, 25 minutes, 20 minutes, 15 minutes, 12 minutes, 10 minutes, 8 minutes, 5 minutes, 2 minutes, or 1 minutes. In some embodiments, all of the liquid carrier may be passed through a filter in about 1-30 minutes, 1-25 minutes, 1-20 minutes, 1-15 minutes, 1-10 minutes, 1-5 minutes, 5-30 minutes, 5-25 minutes, 5-20 minutes, 5-15 minutes, 5-10 minutes, 10-30 minutes, 10-25 minutes, 10-20 minutes, or 10-15 minutes. The time in which the liquid carrier can be passed through a filter may be at least partially driven by a fluidic controller.

In some embodiments, a single cell may be passed through a filter for about 1 microsecond to 10 milliseconds. For example, a cell may be passed through a filter within 1 μs-1 ms, 10 μs-10 ms, 10 μs-1 ms, 100 μs-10 ms, or 100 μs-1 ms. In some embodiments, a cell may be passed through a filter in greater than or equal to about 1 μs, 5 μs, 10 μs, 50 μs, 100 μs, 500 μs, 1 ms, or 5 ms. In some embodiments, a cell may be passed through a filter in less than or equal to about 5 μs, 10 μs, 50 μs, 100 μs, 500 μs, 1 ms, 5 ms, or 10 ms.

In some embodiments, the speed of cells through a filter to induce cell membrane perturbation may be about 1 m/s. In some embodiments, the speed of cells may be between 10 mm/s and 100 m/s. For example, the speed of cells may be less than or equal to 10 mm/s, 20 mm/s, 50 mm/s, 1 m/s, 2 m/s, 5 m/s, 10 m/s, 20 m/s, 50 m/s, or 100 m/s. In some embodiments, the speed of cells may be greater than or equal to 10 mm/s, 20 mm/s, 50 mm/s, 1 m/s, 2 m/s, 5 m/s, 10 m/s, 20 m/s, 50 m/s, or 100 m/s.

### C. Fluid Controllers

The fluid controller described herein control the flow of a liquid carrier from a cell reservoir through the filter to produce the mechanically perturbed cells.

In some embodiments, the fluid controller, in controlling the flow of the liquid carrier from the cell reservoir through the filter, is configured to create (at least in part) a pressure of at least about 1 psi, including about 1 psi to about 50 psi, as measured in the liquid carrier on the upstream adjacent side of the filter. In some embodiments, the fluid controller creates a pressure of about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 21 psi, 22 psi, 23 psi, 24 psi, 25 psi, 26 psi, 27 psi, 28 psi, 29 psi, 30 psi, 31 psi, 32 psi, 33 psi, 34 psi, 35 psi, 36 psi, 37 psi, 38 psi, 39 psi, 40 psi, 41 psi, 42 psi, 43 psi, 44 psi, 45 psi, 46 psi, 47 psi, 48 psi, 49 psi, or 50 psi. In some embodiments, the fluid controller creates (such as by way of controlling a pump and/or a valve) a pressure of about 0.1 psi to about 30 psi, such as any of about 0.1 psi to about 25 psi, about 0.1 psi to about 20 psi, about 0.1 psi to about 15 psi, about 0.1 psi to about 10 psi, about 0.1 psi to about 9 psi, about 0.1 psi to about 8 psi, about 0.1 psi to about 7 psi, about 0.1 psi to about 6 psi, about 0.1 psi to about 5 psi, about 0.1 psi to about 4 psi, about 0.1 psi to about 3 psi, about 0.1 psi to about 2 psi, about 0.1 psi to about 1 psi. In some embodiments, the fluid controller creates a pressure of about 30 psi or less, such as about any of 25 or less, 20 or less, 15 or less, 10 or less, 9 or less, 8 or less, 7 or less, 6 or less, 5 or less, 4 or less, 3 or less, 2 or less, or 1 or less. In some embodiments, the pressure produced by the fluid controller is tunable and/or can be changed during the course of processing cells as described herein.

In some embodiments, the fluid controller comprises a syringe pump or equivalent mechanical device configured to move a plunger through a barrel. In some embodiments, syringe pump can achieve movement in one or more directions, such back-and-forth along an axis. In some embodiments, the syringe pump moves the liquid carrier at a rate of about 0.5 mL/minute to about 60 mL/minute, including any of 1 mL/minute, 2 mL/minute, 3 mL/minute, 4 mL/minute, 5 mL/minute, 7.5 mL/minute, 10 mL/minute, 12.5 mL/minute, 15 mL/minute, 17.5 mL/minute, 20 mL/minute, 25 mL/minute, 30 mL/minute, 35 mL/minute, 40 mL/minute, 45 mL/minute, 50 mL/minute, 55 mL/minute, or 60 mL/minute.

In some embodiments, the fluid controller comprises a pump, such as a peristaltic pump. In some embodiments, the pump can be controlled to turn on and off at desired times. In some embodiments, the flow produced by the pump can be controlled. In some embodiments, the pump is selected based on a desired flow produced therefrom.

In some embodiments, the fluid controller comprises a solenoid. In some embodiments, the cell reservoir is configured to apply a force to a liquid carrier such that the liquid carrier moves towards a solenoid and a filter. In some embodiments, the solenoid is an electromechanical solenoid. In some embodiments, the solenoid is a pneumatic solenoid. In some embodiments, the solenoid is a proportional solenoid. In some embodiments, the solenoid is configured to complete a closed-to-open-to-closed cycle in about 1 millisecond or more. The solenoid may be provided with instruction to complete this closed-to-open-to-closed cycle based on a characteristic of a sample, or a portion thereof, to be produced. For example, the closed-to-open-to-closed cycle may be based on final sample volume, number of cells to be perturbed, qualities of the filter, or aging thereof, and liquid carrier flow rate. In some embodiments, one closed-to-open-to-closed cycle is used to produce one sample. In some embodiments, two or more closed-to-open-to-closed cycle are used to produce one sample.

In some embodiments, the fluid controller, such as a solenoid, is configured to create sample volumes of about 1 μL to about 5 mL, such as about any of 1 μL, 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 50 μL, 75 μL, 100 μL, 125 μL, 150 μL, 175 μL, 200 μL, 225 μL, 250 μL, 300 μL, 325 μL, 350 μL, 375 μL, 400 μL, 425 μL, 450 μL, 475 μL, 500 μL, 600 mL, 700 mL, 800 mL, 900 mL, 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, or 5 mL. In some embodiments, the fluid controller, such as a solenoid, is configured to create a sample volume of 25p L or less. In some embodiments, the fluid controller, such as a solenoid or a syringe pump, is configured to create a sample volume of about 5 μL to about 200 μL, such as any of 5 μL, 6 μL, 7 μL, 8 μL, 9 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 130 μL, 135 μL, 140 μL, 145 μL, 150 μL, 155 μL, 160 μL, 165 μL, 170 μL, 175 μL, 180 μL, 185 μL, 190 μL, 195 μL, or 200 μL. In some embodiments, the high-throughput system is configured such that one or more dispensed volumes make the total sample volume. For example, the sample volume may be from one dispensing cycle of a solenoid or syringe pump. In some embodiments, the sample volume may be the result of two or more dispensing cycles of a solenoid or syringe pump. In some embodiments, the fluid controller, in controlling the flow of the liquid carrier from the cell reservoir through the filter, is configured to pass the liquid carrier through the filter at a volumetric flow rate of between 0.5 mL/min per mm² porous surface area and 500 mL/min per mm² porous surface area. In some embodiments, the fluid controller, in controlling the flow of the liquid carrier from the cell reservoir through the filter, is configured to pass the liquid carrier through the filter at a volumetric flow rate of about 0.5-100, 0.5-10, 50-500, 5-100, or 5-20 mL/min per mm² porous surface area. In some embodiments, the fluid controller, in controlling the flow of the liquid carrier from the cell reservoir through the filter, is configured to pass the liquid carrier through the filter at a volumetric flow rate greater than or equal to about 0.5, 1, 2, 5, 10, 15, 20, 30, 40, 50, 75, 100, 125, 150, 175, 200, 250, 300, 350, 400, or 450 mL/min per mm² porous surface area. In some embodiments, the fluid controller, in controlling the flow of the liquid carrier from the cell reservoir through the filter, is configured to pass the liquid carrier through the filter at a volumetric flow rate less than or equal to about 1, 2, 5, 10, 15, 20, 30, 40, 50, 75, 100, 125, 150, 175, 200, 250, 300, 350, 400, 450, or 500 mL/min per mm² porous surface area.

The pumps described herein may be configured to provide a pressure (e.g., a pulsed pressure, a constant pressure, etc.) to the system between at least 2 psi and 35 psi. For example, the pump may provide a pressure between at least 2-20 psi, 2-10 psi, 10-35 psi, or 10-20 psi. In some embodiments, the pressure provided by pump may be greater than or equal to about 1 psi, 5 psi, 10 psi, 15 psi, 20 psi, 25 psi, 30 psi, or 35 psi. In some embodiments, the pressure provided by pump may be less than or equal to about 1 psi, 5 psi, 10 psi, 15 psi, 20 psi, 25 psi, 30 psi, or 35 psi. In some embodiments, the system may comprise one or more components (e.g., one or more valves, pressure dampeners, etc.) configured to control or modify (e.g., step-down) the pressure provided by the pump. In doing so, a pressure gradient across the filter may be maintained within the intended range (e.g., between 2 psi and 20 psi). In some examples, the pressure gradient across the filter may be less than 2 psi, such as 1.5 psi, 1 psi, 0.5 psi, or 0.25 psi. In some embodiments, the pressure gradient across the filter may be greater than 15 psi, such as 17.5 psi, 20 psi, 22.5 psi, 25 psi, 30 psi, or more. In some examples, the pressure gradient across the filter, such as a silicon filtering surface, may be less than 20 psi, such as 18 psi, 15 psi, 14 psi, 12 psi, 10 psi, 8 psi, 5 psi, or psi. In some examples, the pressure gradient across the filter, such as a silicon filtering surface, may be between 2-20 psi, 5-20 psi, 8-20 psi, or 10-20 psi. In some examples, the pressure gradient across the filter, such as a silicon filtering surface, may be less than 2 psi, such as 1.5 psi, 1 psi, 0.5 psi, or 0.25 psi. In some embodiments, the pressure gradient across the filter, such as a silicon filtering surface, may be greater than 15 psi, such as 17.5 psi, 20 psi, 22.5 psi, 25 psi, 30 psi, or more. The filters described herein can be configured such that at the above-described low-pressure gradients, and using the flow rates described herein, the filtering surface can effectively perturb cell membranes for payload delivery.

The pump may be configured to pump (e.g., move) a total volume between at least 1 mL and 500 mL through the system or a portion thereof in a single run. For example, the pump may be configured to pump between about 1-100 mL, 1-50 mL, 10-500 mL, 10-100 mL, or 100-500 mL through the system in a single run. In some embodiments, the pump may be configured to drive greater than or equal to about 1 mL, 50 mL, 100 mL, 200 mL, 300 mL, 400 mL, or 500 mL of fluid through the system. In some embodiments, pump may be configured to drive less than or equal to about 1 mL, 50 mL, 100 mL, 200 mL, 300 mL, 400 mL, or 500 mL of fluid through the system. In some embodiments, the 436 may be configured to pump less than 1 mL of fluid through the system in a single run, such as a minimum volume of 100 μL, 250 μL, 500 μL, or 750 μL. In some embodiments, the pump may be configured to pump greater than 500 mL of fluid through the system in a single run, such as about 1 L, 2 L, 3 L, 4 L, or 5 L. In some embodiments, the system as described herein may be adapted for allogeneic production and/or bioprocessing, and thus the pump may be configured to pump a volume greater than 5 L, such as 10 L, 100 L, or 1000 L.

### D. Distributors

The distributors described herein comprise one or more dispensing tips for dispensing a liquid carrier comprising mechanically perturbed cells into two or more samples.

Both manual and automated liquid handling, and tools and systems therefor, is well-known in the field. In some embodiments, the one or more dispensing tips of the distributor are pipette tips, including replaceable pipette tips. In some embodiments, the pipette tip comprises a metal, such as stainless steel. In some embodiments, the pipette tip comprises a polymer, such as a plastic.

In some embodiments, the distributor comprises a single dispensing tip. In some embodiments, the distributor comprises a gantry. In some embodiments, the gantry comprises a number of dispensing tips based on the number of wells in a multi-welled plate (e.g., matching the number of wells in a multi-welled plate).

In some embodiments, the one or more dispensing tips of the distributor are configured for non-contact liquid dispensing (also known as contactless liquid dispensing). In such embodiments, such systems may or may not comprise replacement dispensing tips and/or a cleaning station for the dispensing tips.

### E. End-Effectors and Cartridges Comprising the Same

Provided herein, in certain aspects, are end-effectors comprising one or more components of the system. For example, in some embodiments, the end-effector comprises a filter and a fluid controller (such as a solenoid). In some embodiments, the end-effector comprises a filter, a fluid controller, and a dispenser. In some embodiments, the end-effector is configured as a replaceable component (such as in a cartridge) of a system provided herein. In some embodiments, the end-effector can be fluidically coupled to a cell reservoir (such as via suitable tubing and connectors). In some embodiments, the end-effector is configured to engage with an arm (such as a robotic arm) of a liquid handling system.

In some embodiments, the filter, the fluid controller, and the distributor are configured as an end-effector comprising a solenoid positioned upstream and in fluidic communication with a filter positioned upstream and in fluidic communication with the one or more dispensing tips.

In some embodiments, the end-effector is a removable and replaceable cartridge.

In some embodiments, provided herein is a cartridge comprising one or more components described herein. In some embodiments, the cartridge comprises a filter comprising a plurality of pores therethrough for perturbing cell membranes of a plurality of cells. In some embodiments, the cartridge comprises a filter and a distributor comprising one or more dispensing tips. In some embodiments, the cartridge comprises a filter and a fluid controller. In some embodiments, the cartridge comprises a fluid controller and a distributor. In some embodiments, the cartridge comprises a filter, a fluid controller, and a distributor. In some embodiments, the high-throughput system comprises one or more cartridges (such as for providing a plurality of a single component, e.g., a high-throughput system with two or more filters, and/or providing a plurality of different components, e.g., a filter and a fluid controller). In some embodiments, the cartridge is configured as a removable cartridge. In some embodiments, the cartridge is configured as a removable and replaceable cartridge.

In some embodiments, the high-throughput system and/or an aspects of one or more components, such as a cartridge, filter, fluid controller (e.g., solenoid), or distributor, comprises a mount for attaching said one or more components to a high-throughput liquid handler system.

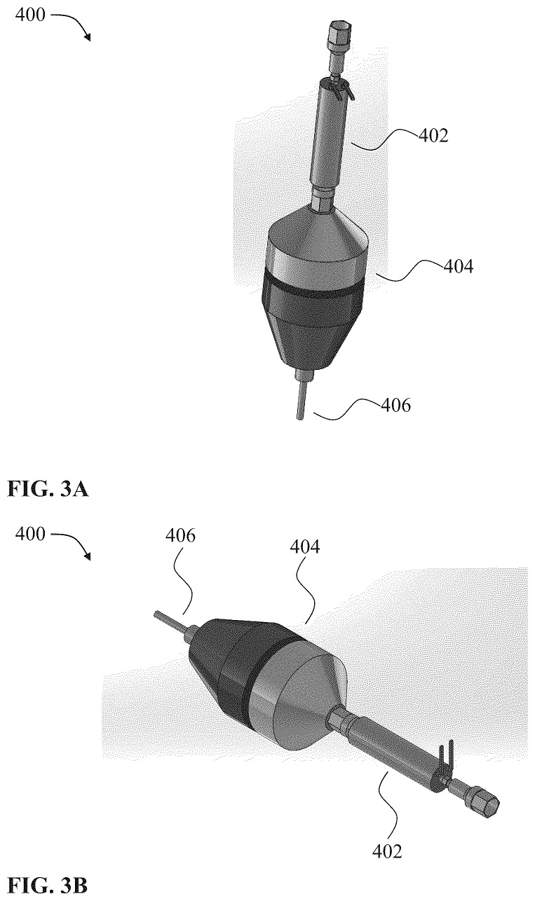

As shown in FIGS. 3A and 3B, provided is an end-effector 400 comprising a solenoid 402, a housing holding a filter 404 and a distributor comprising a dispensing tip 406. A liquid carrier comprising cells can be passed through the filter 404 and out the dispensing tip 406 by controlling the opening and closing of the solenoid 402. Such end-effector 400 can be attached to a flow of liquid carrier, e.g., from a pressurized cell reservoir.

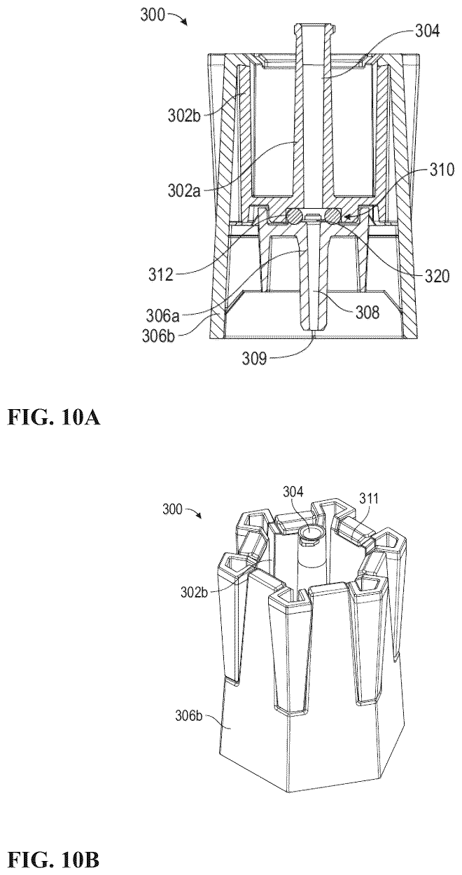

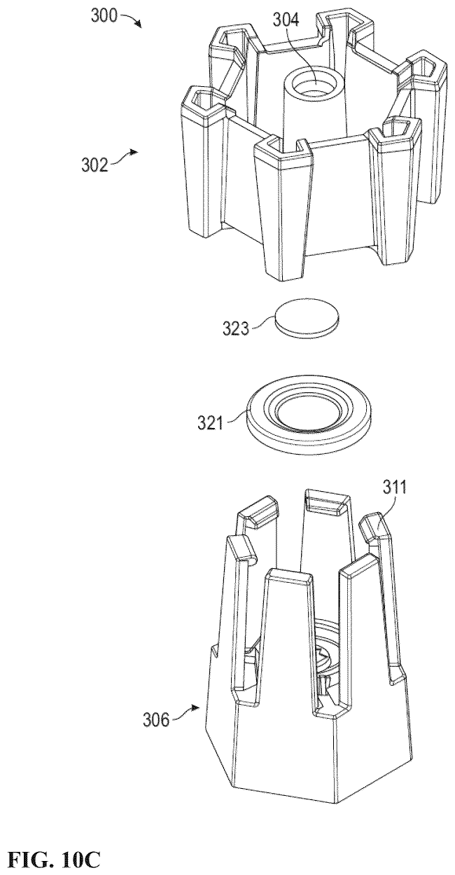

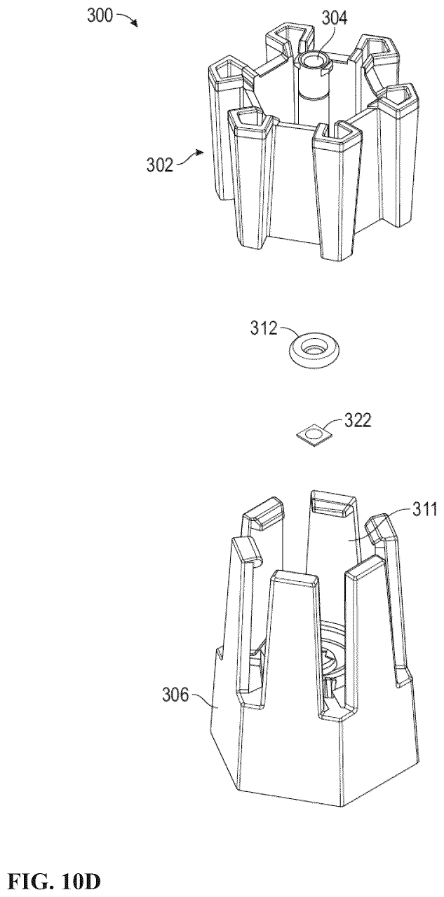

As shown in FIGS. 10A-10D, the cartridge 300 may have a larger external structure (e.g., housing) that can enable the user to easily grip the cartridge 300. By creating a larger external housing around the fluid inlet 304, fluid outlet 308, and the filter 320 held therebetween, the cartridge 300 can more easily be handled. For example, the user can grip the cartridge 300 to insert and remove the cartridge 300 from a high-throughput system described herein. The external structure of the cartridge 300 can be composed of the outer first housing portion 302b and the outer second housing portion 306b, each of which are described below. The first housing portion 302 can include an inner portion (302a) and an outer portion (302b). The inner portion 302a can surround at least the fluid inlet 304 and the filter holding portion 310, as shown in FIG. 10A. The outer portion 302b can comprise the remainder of the first housing portion 302, including the portion which the user engages with to handle the cartridge 300. The cartridge 300 may comprise voids, or spaces, between the inner and outer portions 302a, 302b of the first housing portion 302, at least to minimize the weight and/or improve usability of the cartridge 300. The second housing portion 306 can also include an inner portion (306a) and an outer portion (306b). The inner portion 306a can surround at least the fluid outlet 308 and the filter holding portion 310, as shown in FIG. 10A. The outer portion 306b can comprise the remainder of the second housing portion 306, including the portion which the user can engage with to handle the cartridge 300. Similar to the first housing portion 302, the second housing portion 306 can comprise voids between the inner and outer portions 306a, 306b of the second housing portion 306, at least to minimize the weight and/or usability of the cartridge 300. In cartridge 300, the first housing portion 302 may removably connect with the second housing portion 306 to enable a user to operably connect and disconnect the housing portions (e.g., to insert and/or remove filter 320 from within the cartridge 300). As illustrated in FIG. 10B, the outer second housing portion 306b can include one or more latches 311 configured to removably connect the housing portions 302b, 306b. The latches may be disposed circumferentially around the fluid inlet 304 and fluid outlet 308. As illustrated in FIG. 10A, the inner second housing portion 306b may be insertable to a receptacle of inner first housing portion 306a (or vice versa), for example, to stabilize the connection between the housing portions. In some embodiments, as illustrated in the exploded view of cartridge 300 in FIG. 10C, the cartridge 300 may hold a mesh support 321 within the first housing portion 302 and second housing portion 306. The mesh support 321 may be usable with a polymer filter 320 to support the filter. In some examples, the mesh support 321 can act as the sealing component 320 in the earlier described embodiments The cartridge 300 may comprise a cell mechanoporation filter 320 held by the first housing portion 302 and the second housing portion 306. A sealing component 312 may be disposed between the first housing portion 302 and the second housing portion 306 to seal the connection between the two housing portions holding the filter 320. The fluid outlet 308 may be removably connectable to the output reservoir by disposing the fluid outlet proximate to an opening of the output reservoir. The fluid outlet 308 may comprise a constriction 309 at a distal end of the fluid outlet 308 that can direct the fluid through the fluid outlet 308 and into the output reservoir while minimizing any sample waste. The constriction 309 can comprise a width (e.g., diameter) that is at least 20% less than the width (diameter) of the fluid outlet 308. For example, the width of the constriction 309 may be at least 20%, 30%, 40%, 50%, 60%, 70%, or 80% less than the width of the fluid outlet 308. In some embodiments, the cell mechanoporation filter 120 (and filter 220, collectively referred to herein as filter 120) may comprise a silicon filter or a polymer filter. For example, FIG. 3D depicts an exemplary cartridge 300 comprising a silicon filter 322.

### F. Cell Reservoirs

The cell reservoirs described herein are configured to hold a liquid carrier comprising a plurality of cells. The cell reservoirs may be integrated to the systems described herein such that the plurality of cell (prior to mechanical perturbation) are accessible to other components and are maintained in a suitable manner (such as at an appropriate condition, e.g., temperature, for a cell).

The cell reservoirs taught herein may be configured to hold a wide range of volumes, including volumes for a liquid carrier comprising a plurality of cells. In some embodiments, the cell reservoir is configured to be coupled with a component provided pressurization thereof. Thus, in some embodiments, the cell reservoir is of construction and material suitable for cells and such forces and environments. In some embodiments, the cell reservoir has a volume to hold liquid carrier of about 1 mL to about 100 mL, such as about 1 mL to about 5 mL. In some embodiments, the cell reservoir is integrated with another component described herein, such as the cell reservoir being a barrel, or portion thereof, of a syringe.

In some embodiments, the cell reservoir comprises a fluid reservoir, flexible plastic bag, vial, bottle, vessel, ampule, jar, or other container suitable for containing the cell mixture. In another example, the cell reservoir may not comprise a separate vessel containing the liquid carrier, but rather the system is configured to receive the liquid carrier from an upstream, fluidly connectable cell therapy production system, cell washing system, filtration system, etc. In some embodiments, one or more of the aforementioned systems may process cells prior to providing the mixture to a system by surface marker-based separation (e.g., Miltenyi), centrifugation/flow-based separation (e.g., elutriation), buffer exchanges, cell washing, activations, expansions, or other biological variations.

Example filtration and/or elutriation systems that may be fluidly connectable to the system may include but are not limited to tangential flow filtration (TFF) systems, (e.g., Repligen KrosFlo® KR2i TFF system, Sartorius Ambr® Crossflow TFF system, Sartorius Sartoflow® Smart TFF system, etc.), standard filtration systems (e.g., Fresenius Cue® Cell Processing System), spinning membrane filtration, (e.g., Fresenius Kabi Lovo® Automated Cell Processing System), and elutriation systems (e.g., Gibco™ Cell Therapy Systems (CTS™) Rotea Counterflow Centrifugation System, Terumo Elutra® Cell Separation System, etc.). Example cell isolation systems include but are not limited to the Miltenyi CliniMACS Prodigy® instrument and StemCell cell isolation systems (e.g., immunomagnetic cell separation systems such as EasySep™, RoboSep™ and StemSep™, immunodensity cell separation systems such as RosetteSep™ and SepMate™, etc.). Example end-to-end cell therapy production systems include but are not limited to the Lonza Cocoon® Platform.

In some embodiments, the cell reservoir is pressurized such that a force is applied to a liquid carrier therein in a manner that liquid carrier moves towards a filter. In some embodiments, the pressurization of the cell reservoir is a positive pressure. In some embodiments, the cell reservoir is pressurized with a gas. In some embodiments, the gas is air, CO₂, an inert gas (such as nitrogen), or any mixture thereof. In some embodiments, the cell reservoir is pressurized with a pump. In some embodiments, the pump is a peristaltic pump. In some embodiments, the pressurization of the cell reservoir is a negative pressure.

In some embodiments, the cell reservoir, such as the pressurized cell reservoir, is configured to transport the liquid carrier to a filter via tubing. In some embodiments, the high-throughput liquid handler system comprises tubing to fluidically connect a cell reservoir, such as a pressurized reservoir, and a filter. In some embodiments, the fluidic connection between a cell reservoir and a filter may comprise additional components, in addition to tubing, along the fluid path from the cell reservoir and the filter. The tubing taught herein can be characterized by way of an inner diameter (ID) and/or a length. In some embodiments, the ID and length of tubing may be selected based on the ID and length and the pressure drop as measured from the end of the tubing closest to a cell reservoir to the end of the tubing closest to the filter. For example, in some embodiments, the ID and length of a tubing are selected such that the pressure drop from the end of the tubing closest to a cell reservoir to the end of the tubing closest to the filter is about 10 psi or less, such as about any of 9 psi or less, 8 psi or less, 7 psi or less, 6 psi or less, 5 psi or less, 4 psi or less, 3 psi or less, 2 psi or less, or 1 psi or less. In some embodiments, the inner diameter (ID) of the tubing is about 0.1 mm to about 10 mm, such as any of about 0.5 mm to about 1 mm, about 0.5 mm to about 2.5 mm, about 1 mm to about 3 mm, about 1 mm to about 8 mm, about 1.5 mm to about 5 mm, or about 5 mm to about 10 mm. In some embodiments, the inner diameter of the tubing is about any of 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, or 10 mm. In some embodiments, the tubing is characterized by way of a length, as measured from the distanced covered between a reservoir and a filter. In some embodiments, the length of the tubing is about 10 mm to about 1,000 mm, such as about any of 200 mm to about 450 mm or about 250 mm to about 400 mm. In some embodiments, the length of the tubing is about any of 10 mm, 25 mm, 50 mm, 75 mm, 100 mm, 150 mm, 200 mm, 250 mm, 300 mm, 350 mm, 400 mm, 450 mm, 500 mm, 550 mm, 600 mm, 650 mm, 700 mm, 750 mm, 800 mm, 850 mm, 900 mm, 950 mm, or 1,000 mm. The tubing envisioned herein is compatible with the conditions and components of use described herein, e.g., suitable for use with cells and capable of withstanding certain pressures. In some embodiments, the tubing comprises Tygon PVC, PTFE, silicone rubber tubing, e.g., with a durometer of about 40 A to about 60 A. In some embodiments, the tubing has a length that is not more than about 200× of the inner diameter of the tube, including not more than about any of 175×, 150×, 125×, 100×, 75×, 50×, or 25× of the inner diameter of the tube.

### G. Automation

The high-throughput systems described herein are amenable to various degrees of automation, including full automation once a liquid carrier comprising cells is placed in a cell reservoir. High-throughput liquid handlers are well known in the field, and components of the high-throughput systems described herein can be integrated therewith to provide automation. Additionally, the high-throughput systems described comprise controllers, such as those including computers and user-interfaces, to allow a method of distributing mechanically perturbed cells to be performed.

In some embodiments, the high-throughput system comprises a commercially available liquid handler (or a portion thereof). For examples, liquid handlers may be acquired from GNF, Tecan, Hamilton Robotics, Eppendorf, Opentrons, Automata, Agilent, TriContinent, Perkin Elmer, and/or Beckman Coulter. Such liquid handlers may be modified to include the components taught herein.

In some embodiments, the high-throughput system comprises one or more arms, such as a robotic arm, the one or more arms separately connected, directly or indirectly, to one or more of a filter, a fluid controller, or a distributor. The present description encompasses a multitude of configurations of arms and robotic arms, and one of ordinary skill in the art will readily appreciate which components need to move relative to one another to perform distribution of mechanically perturbed cells. For example, in some embodiments, the high-throughput system comprises an end-effector attached to a robotic arm, wherein the robotic arm can place a dispensing tip of the end-effector in such a position as to distribute samples to a multi-welled plate. In some embodiments, the high-throughput system comprises a stationary end-effector (which may be attached to stationary an arm), wherein multi-welled plate is on a sample holder configured to move such that produced samples from a dispensing tip of the end-effector are distributed to a multi-welled plate.

In some embodiments, the robotic arm comprises an x- and y-plane positioner. In some embodiments, the robotic arm comprises an x-, y-, and z-plane positioner.

In some embodiments, the high-throughput liquid handler is communicatively couplable to a control system to control one or more operating parameters of the high-throughput liquid handler. In some embodiments, the high-throughput liquid handler comprises a control system communicatively coupled to the high-throughput liquid handler to control one or more operating parameters of the high-throughput liquid handler. In some embodiments, the control system comprises: one or more processors; and memory storing one or more programs, the one or more programs configured to be executed by the one or more processors, the one or more programs including instructions for at least one of, e.g., controlling the flow the liquid carrier; opening a solenoid; closing the solenoid; controlling fluid volume in a syringe; positioning one or more robotic arm; or position a sample holder.

In some embodiments, the high-throughput liquid handler comprises one or more sensors to facilitate operation thereof, including automated operation. In some embodiments, wherein the high-throughput liquid handler comprises a collection well, the high-throughput liquid handler comprises an infrared (IR) sensor, e.g., and IR beam sensor, configured to detect when a distributor comprising one or more dispensing tips, or a portion thereof, is in proximity to a collection well. For example, in some embodiments, the high-throughput liquid handler comprises an IR sensor and beam located above a collection well such that when one or more dispensing tips of a distributor are in close proximity to the collection well the handler will then pass a cell solution through a filter and mechanoporated cells are collected in the collection well. The distributor can then deliver aliquots of the mechanoporated cells as desired within a desired residency time. In some embodiments, the high-throughput liquid handler is configured such that there is a timing delay to prevent subsequent triggering of an IR sensor following an initial IR sensor trigger.

In some embodiments, wherein the high-throughput liquid handler comprises a collection well, the high-throughput liquid handler is configured to remove any remaining cell solution in a collection after a distributor has collected an aliquot therefrom and prior to additional mechanoporated cells being placed therein. In some embodiments, the high-throughput liquid handler is configured to distinguish a distributor collecting an aliquot from a collection well and the distributor (or another component of the handler) collecting an aliquot from the collection well for purposes of emptying the collection well.

### H. Collection Wells

In some aspects, a high-throughput system described herein comprises one or more collection wells configured to hold mechanically perturbed cells. In some embodiments, the collection well configured to collect a liquid carrier comprising the mechanically perturbed cells from a filter prior to a distributor dispensing the liquid carrier comprising the mechanically perturbed cells from the collection well into the two or more samples. The collection well encompassed herein may be of any shape and size, and such characteristics may be based on one or more attribute of a high-throughput system or method performed therewith. For example, a collection well may be sized according to the volume of liquid carrier comprising mechanically perturbed cells intended to be processed, and/or the volume of liquid to be held in the collection well. A collection well may be sized according to a distributor, e.g., accommodate a multi-channel pipette.

As described herein, in some embodiments, fluid, e.g., a liquid carrier comprising cells and/or a wash, deposited in a collection well is removed therefrom. In some embodiments, the high-throughput liquid handler is configured to remove a fluid from a collection well using a distributor comprising one or more dispensing tips. In some embodiments, the high-throughput liquid handler is configured to remove a fluid from a collection well using a distributor comprising one or more tips dedicated for use with the collection well. In some embodiments, the high-throughput liquid handler is configured to remove a fluid from a collection well via a drain located thereon. In some embodiments, the drain of a collection well is configured to be controllable in regards to the timing of opening and closing the drain, such as via a controllable valve. In some embodiments, the high-throughput liquid handler is configured to apply a wash solution to the collection well, such as prior to depositing a mechanoporated cell therein.

### I. Sample Holders and Sample Plates

In some aspects, the high-throughput system comprises a sample holder. In some embodiments, the sample holder is configured to secure a multi-well plate having 2 or more wells to receive two or more samples. In some embodiments, the sample holder comprises an x- and y-plane positioner. In some embodiments, the sample holder comprises an x-, y-, and z-plane positioner.

As described herein, the high-throughput systems comprise a distributor comprising one or more dispensing tips for dispensing a liquid carrier comprising mechanically perturbed cells into two or more samples. The samples described herein may be separated by a multitude of ways, including separate container, multi-welled plates, or distinct droplets. General description is provided herein for sample plates, however, it is to be understood that such description can be applied to equivalent means for maintaining separation between produced samples of mechanically perturbed cells. In some embodiments, the multi-well plate comprises 2 or more wells. In some embodiments, the multi-well plate comprises 96 wells. In some embodiments, the multi-well plate comprises 384 wells. In some embodiments, the multi-well plate comprises 1536 wells.

The sample sizes produced by the high-throughput liquid handler described herein may be varied as desired. In some embodiments, the sample size produced is about 1 μL to about 300 μL, such as about any of 1 μL, 2 μL, 3 μL, 4 μL, 5 μL, 6 μL, 7 μL, 8 μL, 9 μL, 10 μL, 15 μL, 20 μL, 25 μL, 30 μL, 45 μL, 50 μL, 60 μL, 70 μL, 80 μL, 90 μL, 100 μL, 125 μL, 150 μL, 175 μL, 200 μL, 225 μL, 250 μL, 275 μL, or 300 μL.

In some embodiments, the sample well, such as a sample well of a multi-welled plate, is configured based on the volume of liquid to be held therein. The collection well encompassed herein may be of any shape and size, and such characteristics may be based on one or more attribute of a high-throughput system and/or method performed therewith. In some embodiments, the sample well comprises a feature useful in a cellular assay, such as a coating or a surface suitable for a read-out, such as imaging or fluorescence measurement.

### J. Liquid Carriers and Cells

As described herein, the cells are suspending in a liquid carrier when passed through a filter comprising a plurality of pores therethrough. Thus, in some embodiments, provided herein is a high-throughput system comprising a liquid carrier comprising a plurality of cells.

In some embodiments, the liquid carrier has a volume of about 10 μL to about 5 L, such as any of 100 μL, 500 μL, 1 mL, 5 mL, 10 mL, 50 mL, 100 mL, 200 mL, 300 mL, 400 mL, 500 mL, 600 mL, 700 mL, 800 mL, 900 mL, 1 L, 2 L, 3 L, 4 L, or 5 L. In some embodiments, the liquid carrier has a volume of at least about 10 μL, such as at least about any of 100 μL, 500 μL, 1 mL, 5 mL, 10 mL, 50 mL, 100 mL, 200 mL, 300 mL, 400 mL, 500 mL, 600 mL, 700 mL, 800 mL, 900 mL, 1 L, 2 L, 3 L, 4 L, or 5 L. Accordingly, in some embodiments, the methods comprise selecting a mechanical deformation technique, or component thereof such as a filter, suitable for processing such volume.

In some embodiments, the liquid carrier has a cell density of at least about 100 cells/mL, such as at least about 200 cells/mL, 300 cells/mL, 400 cells/mL, 500 cells/mL, 600 cells/mL, 700 cells/mL, 800 cells/mL, 900 cells/mL, 1,000 cells/mL, 1,250 cells/mL, 1,500 cells/mL, 1,750 cells/mL, 2,000 cells/mL, 2,250 cells/mL, 2,500 cells/mL, 2,750 cells/mL, 3,000 cells/mL, 3,250 cells/mL, 3,500 cells/mL, 3,750 cells/mL, 4,000 cells/mL, 4,250 cells/mL, 4,500 cells/mL, 4,750 cells/mL, 5,000 cells/mL, 10,000 cells/mL, 50,000 cell/mL, 100,000 cell/mL, 1,000,000 cell/mL, 5,000,000 cells/mL, 10,000,000 cells/mL, 25,000,000 cells/mL, 50,000,000 cells/mL, 100,000,000 cells/mL, 150,000,000 cells/mL, or 200,000,000 cells/mL. In some embodiments, the liquid carrier has a cell density of about 1,000,000 cells/mL to about 200,000,000 cells/mL.

In some embodiments, the liquid carrier comprises between about 1.0×10⁸ and 1.0×10¹² cells. In some embodiments, the liquid carrier comprises between about 1.0×10⁸ and 1.0×10¹¹ cells, 1.0×10⁸ and 1.0×10¹⁰ cells, or 1.0×10⁶ and 1.0×10⁹ cells. In some embodiments, the liquid carrier comprises greater than or equal to about 1.0×10⁸ cells, 5.0×10⁸ cells, 1.0×10⁹ cells, 5.0×10⁹ cells, 1.0×10¹⁰ cells, 5.0×10¹⁰ cells, 1.0×10¹¹ cells, or 5.0×10¹¹ cells. In some embodiments, the liquid carrier comprises less than or equal to about 5.0×10⁸ cells, 1.0×10⁹ cells, 5.0×10⁹ cells, 1.0×10¹⁰ cells, 5.0×10¹⁰ cells, 1.0×10¹¹ cells, 5.0×10¹¹ cells, or 1.0×10¹² cells.

In some embodiments, the high-throughput system processes a volume of a liquid carrier through a filter comprising at least about 1 million cells (such as about 1 million to 500 million cells), such as processes the volume using a single filter. In some embodiments, the high-throughput system processes a volume of liquid carrier through a filter comprising between about 1.0×10⁸ and 1.0×10¹² cells. In some embodiments, the high-throughput system processes a volume of liquid carrier through a filter comprising between about 1.0×10⁸ and 1.0×10¹¹ cells, 1.0×10⁸ and 1.0×10¹⁰ cells, or 1.0×10⁶ and 1.0×10⁹ cells. In some embodiments, the high-throughput system processes a volume of liquid carrier through a filter comprising greater than or equal to about 1.0×10⁸ cells, 5.0×10⁸ cells, 1.0×10⁹ cells, 5.0×10⁹ cells, 1.0×10¹⁰ cells, 5.0×10¹⁰ cells, 1.0×10¹¹ cells, or 5.0×10¹¹ cells. In some embodiments, the high-throughput system processes a volume of liquid carrier through a filter comprising less than or equal to about 5.0×10⁸ cells, 1.0×10⁹ cells, 5.0×10⁹ cells, 1.0×10¹⁰ cells, 5.0×10¹⁰ cells, 1.0×10¹¹ cells, 5.0×10¹¹ cells, or 1.0×10¹² cells.

In some embodiments, the cell is a cell obtained from, or derived from, an individual. In some embodiments, the individual is a mouse. In some embodiments, the individual is a non-human primate. In some embodiments, the individual is a human. In some embodiments, the individual is a mouse, dog, cat, horse, rat, goat or rabbit.

In some embodiments, the liquid carrier substantially contains a single cell type (e.g., at least about 90% of the cells in a liquid carrier are of a single cell type, e.g., T cells). In some embodiments, the liquid carrier comprises a plurality of cell types, e.g., as found in cells from whole blood, lymph, and/or peripheral blood mononuclear cells (PBMCs). In some embodiments, the liquid carrier comprises a purified cell population.

In some embodiments, the cell (or cells) is a somatic cell, or a stem cell, or an immortalized cell, or a derivative thereof. In some embodiments, the cell is an immune cell, or derivative thereof. In some embodiments, the cell (or cells) is a PBMC, or a derivative thereof. In some embodiments, the cell (or cells) is a T cell, NK cell, monocyte, B cell, or dendritic cell (or mixture thereof), or derivative thereof. In some embodiments, the cell is an unstimulated T cell, or derivative thereof. In some embodiments, the cell is a stimulated (or activated) T cell. In some embodiments, the cell is a stem cell, or derivative thereof. In some embodiments, the cell is a human stem cell, or derivative thereof. In some embodiments, the cell is a hematopoietic stem cell (HSC), or derivative thereof. In some embodiments, the cell is a mesenchymal stem cell (MSC), or derivative thereof. In some embodiments, the cell is an induced pluripotent stem cell, or derivative thereof. In some embodiments, the cell is a cancer cell line, such as a HEK293, or derivative thereof. In some embodiments, the immortalized cell is a cancer cell.

In some embodiments, the liquid carrier comprises a buffer. Such buffer may, in certain aspects, serve as a vehicle to enable processing of cells as described herein. As the buffer will contain cells, buffers envisioned herein are compatible with the cell being processed, such as, e.g., to avoid lysis and/or undesirable alterations of said cell. For example, in some embodiments, the buffer has a desired osmolarity, salt concentration, serum content, cell concentration, and pH. In some embodiments, the buffer is a physiological saline solution or a physiological medium other than blood.

In some embodiments, the liquid carrier comprises an aqueous solution. In some embodiments, the aqueous solution comprises cell culture medium, PBS, salts, sugars, growth factors, animal derived products, bulking materials, surfactants, lubricants, vitamins, proteins, chelators, and/or an agent that impacts actin polymerization. In some embodiments, the cell culture medium is DMEM, Opti-MEM, EVIDM, RPMI, or X-VIVO. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

In some embodiments, the cells can be incubated in one or more solutions that aid in the delivery of the cargo to the interior of the cell (such as by keeping the formed pores in the cell membranes open for longer periods of time). In some embodiments, the aqueous solution comprises an agent that impacts actin polymerization. In some embodiments, the agent that impacts actin polymerization is Latrunculin A, Cytochalasin, and/or Colchicine. In some embodiments, the cell can be incubated in a liquid carrier comprising a desired level of calcium (including no or substantially no calcium, e.g., less than 1 mM). In some embodiments, the temperature experienced by the cell is controlled such as to control the speed at which the pores formed in the cells close, e.g., by using lower temperatures such as between 10° C. and 0° C.

In some embodiments, the cells are in a first buffer (such as when placed in a cell reservoir) and the payload is in a second buffer (such as when placed in a sample well), wherein the first buffer and the second buffer are different. In some embodiments, the first buffer may promote pores in the mechanically perturbed cells to remain open for a longer period of time as compared to the same cells without said first buffer, e.g., PBS. In some embodiments, the second buffer may induce pores in the mechanically perturbed cells to close and/or promote cell health following mechanical perturbation, e.g., Optimem.

In some embodiments, the liquid carrier comprises one or more payloads to be delivered to the mechanically perturbed cells. Such payloads are in addition to the payload introduced to mechanically perturbed cells following distribution by a high-throughput system. Such additional payloads may be helpful in situations when a universal payload is desired to also be introduced to two or samples.

### K. Payloads and Efficacy of Delivery

As described herein, the mechanically perturbation of cell is for intracellular delivery of one of more payloads, wherein at least one of the payloads is introduced by distribution of a mechanically perturbed cell sample to the at least one payload. In some embodiments, one or more payload is present at the time of passing a cell through a filter such that the one or more payloads are delivered to a cell, such as described as universal payload in other section herein. For example, such configuration enables the high-throughput production of separate population of cells having been subjected to intracellular delivery of A+B, A+C, A+D, and so on (where A was present with the cell as it passed through the filter for mechanically perturbation).

In some embodiments, the one or more payloads are individually selected from the group consisting of a polypeptide, a nucleic acid, a small molecule compound, and a polymer, or a mixture thereof. In some embodiments, the polypeptide is a protein. In some embodiments, the payload is a gene editing complex.

The type of payload as well as the concentration thereof may be dependent on the type of cells in the liquid carrier and the intended cellular assay. For example, the payload may include but is not limited to one or more of DNA, RNA (e.g., mRNA, siRNA, saRNA, tRNA, miRNA, circular RNA, etc.), proteins, small molecules (e.g., a therapeutic candidate or a precursor thereof), peptides, nanoparticles, viruses, synthetic materials, a cell lysate comprising an antigen, and/or complexes (e.g., RNP, protein CAS plus guide RNA, etc.). The payload may comprise a plurality of payloads (e.g., a mixture of nucleic acids, proteins, small molecules, etc.) The aforementioned payloads may function biologically as transcription factors, chemokines, cytokines, surface receptors, other intracellular or extracellular proteins, inhibitors or enhancers of any of the above, survival factors, cryoprotectants, prime editors, antibodies, enzymes, and/or any combinations of the above. In some embodiments, the payload comprises a fluorophore, including a fluorophore label attached thereto. For example, in some embodiments, the payload is a polypeptide comprising a fluorescent label, e.g., a fluorescein isothiocyanate (FITC) label. In some embodiments, the payload is a DNA-encoded library (DEL; in some embodiments also referred to as a DNA-encoded chemical library) (or a single species thereof).

In some embodiments, the payload may comprise at least one molecule having a molecular weight between about 100 Da and 10 MDa. For example, as described herein, the payload may comprise small molecules (˜100 Da) and/or large plasmids (˜10 MDa). For example, a molecular weight of a molecule in the payload may be between about 100 Da and 1 MDa, 100 Da and 100 kDa, or 100 Da and 1 kDa. In some embodiments, the payload may comprise at least one molecule having a molecular weight greater than or equal to about 100 Da, 500 Da, 1 kDa, 50 kDa, 100 kDa, 500 kDa, 1 MDa, or 5 MDa. In some embodiments, the payload may comprise at least one molecule having a molecular weight less than or equal to about 500 Da, 1 kDa, 50 kDa, 100 kDa, 500 kDa, 1 MDa, 5 MDa, or 10 MDa. As described herein, the payload may comprise a plurality of molecules, particles, etc. having different molecular weights selected from those listed above.

As the mechanoporation mechanism described herein is dependent on the payload transfecting into a pore of the cell membrane, the diameter of the payload (e.g., particle, molecule, etc.) may be less than 100 nm. For example, the particle size may be between about 1-100 nm, 1-50 nm, or 1-10 nm. In some embodiments, the particle size (diameter) may be greater than or equal to 1, 5, 10, 25, 50, or 75 nm. In some embodiments, the particle size (diameter) may be less than or equal to 5, 10, 25, 50, 75, or 100 nm. In some embodiments, the particle size is less than 1 nm, such as between about 0.01 nm and about 1 nm.

The concentration of the payload in the liquid carrier or the resulting mixture of the payload when the mechanically perturbed cells are later added may vary based on the type of payload and may be between about 1 nM and 1 mM. For example, the payload concentration may be between about 10 nM-1 mM, 100 nM-1 mM, 1 μm-1 mM, 10 μM-1 mM, or 100 μM-1 mM. In some embodiments, the concentration of the payload may be greater than or equal to 1 nM, 10 nM, 50 nM, 100 nM, 500 nM, 1 μm, 5 μm, 10 μm, 50 μm, 100 μm, 500 μm, or 1 mM. In some embodiments, the concentration of the payload may be less than or equal to 1 nM, 10 nM, 50 nM, 100 nM, 500 nM, 1 μm, 5 μm, 10 μm, 50 μm, 100 μm, 500 μm, or 1 mM.

The payloads may be present in different forms, some of which are dictated by the format of the high-throughput system and point at which the payload is with the cell. In some embodiments, a sample comprising a mechanically perturbed cell may be added to a sample comprising a payload in solution. In some embodiments, a sample comprising a mechanically perturbed cell may be added to a sample comprising a payload in a dried form, such as a dry or lyophilized powder or cake.

In some embodiments, the high-throughput liquid handlers described herein, provide a plurality of cells having a certain degree of delivery efficacy and/or cell viability. Such delivery efficacy can be assessed across wells containing samples prepared by a single filter.

In some embodiments, following passing cells through a filter and subsequent distribution by a high-throughput system described herein at least about 25%, such as at least about any of 30%, 35%, 40%, 45%, 50%, 55%, 60%, 65%, 70%, 75%, 80%, 85%, 90%, or 95%, of the cells receive an amount of one or more payloads above a desired threshold.

In some embodiments, following passing cells through a filter and subsequent distribution by a high-throughput system described herein at least about 25%, such as at least about any of 30%, 35%, 40%, 45%, 50%, 55%, 60%, 65%, 70%, 75%, 80%, 85%, 90%, or 95%, of cells are viable.

In some embodiments, following passing cells through a filter and subsequent distribution by a high-throughput system described herein at least about 50%, such as at least about any of 55%, 60%, 65%, 70%, 75%, 80%, 85%, 90%, 95%, or 100%, of cells receive delivery of a payload.

### L. Additional Aspects

The present disclosure further encompasses components for upstream and/or downstream processes useful or associated with the high-throughput systems described herein.

In some embodiments, the high-throughput system comprises one or more of a well such as for a buffer, reagent, and/or payload (and, in some aspects, the desired item therein), a wash station, such as for washing a dispensing tip, additional dispensing tips, a mixer/agitator, and features to control the environment (such as sterility, temperature, e.g., low temperatures including between 10° C. and 0° C., etc.).

As discussed herein, the distributed mechanically perturbed cells from the high-throughput systems described herein are suitable for downstream uses, including high-throughput cellular assays. It is envisioned herein that the high-throughput systems comprise, or can be coupled with, components useful for one or more aspects of said downstream uses. For example, in some embodiments, the high-throughput system comprises, or can be coupled with, a cell incubator. In some embodiments, the high-throughput system comprises, or can be coupled with, an instrument for a cell assay.

### M. Example High-Throughput Systems

In some embodiments, provided herein is a high-throughput liquid handler configured for distribution of mechanically perturbed cells, the high-throughput liquid handler comprising: a syringe comprising an outlet and a cell reservoir configured to hold a liquid carrier comprising a plurality of cells; a filter placed in fluidic communication with the cell reservoir of the syringe via the outlet, the filter comprising a plurality of pores therethrough for perturbing cell membranes of the plurality of cells; a fluid controller comprising a syringe pump configured to control the flow of the liquid carrier from the cell reservoir through the filter to produce the mechanically perturbed cells; a collection well situated to collect the liquid carrier comprising the mechanically perturbed cells from the filter; and a distributor comprising one or more dispensing tips for dispensing the liquid carrier comprising the mechanically perturbed cells from the collection well into two or more samples. In some embodiments, the distributor comprising the one or more dispensing tips is a gantry.

In some embodiments, provided is a high-throughput liquid handler configured for distribution of mechanically perturbed cells, the high-throughput liquid handler comprising: a syringe comprising an outlet and a filter placed in the fluidic path of the outlet, the filter comprising a plurality of pores therethrough for perturbing cell membranes of the plurality of cells; a cell reservoir configured to hold a liquid carrier comprising a plurality of cells; a fluid controller comprising a syringe pump configured to control the flow of the liquid carrier through the filter such that the liquid carrier comprising the plurality of cells is aspirated from the cell reservoir through the filter and then dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

In some embodiments, provided is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller comprising a solenoid configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples. In some embodiments, the reservoir is pressurized such that a force is applied to the liquid carrier therein in a manner that liquid carrier moves towards the filter. In some embodiments, the pressurization of the reservoir is a positive pressure. In some embodiments, the reservoir is pressurized with a gas. In some embodiments, the gas is air, CO₂, an inert gas, or any mixture thereof. In some embodiments, the reservoir is pressurized with a pump. In some embodiments, the pump is a peristaltic pump. In some embodiments, the filter, the fluid controller, and the distributor are configured as an end-effector comprising a solenoid positioned upstream and in fluidic communication with a filter positioned upstream and in fluidic communication with the one or more dispensing tips. In some embodiments, the end-effector is a removable and replaceable cartridge.

In some embodiments, provided herein is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells is dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples. In some embodiments, the distributor comprising the one or more dispensing tips is a gantry.

In some embodiments, provided herein is a high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells flows through the filter to produce mechanically perturbed cells; and a distributor comprising one or more dispensing tips for dispensing the liquid carrier comprising the mechanically perturbed cells from the syringe into two or more samples. In some embodiments, the distributor comprising the one or more dispensing tips is a gantry.

In some embodiments, provided is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir using tubing, the filter comprising a plurality of pores therethrough for perturbing cell membranes of the plurality of cells; a fluid controller comprising a solenoid configured to control the flow of the liquid carrier from the cell reservoir through the filter to produce the mechanically perturbed cells; and a distributor comprising a dispensing tip (e.g., also called a dispensing nozzle) for dispensing the liquid carrier comprising the mechanically perturbed cells into two or more samples, wherein the high-throughput system is configured such that the liquid carrier travels from the cell reservoir through the filter then through the solenoid prior to being dispensed out of the dispensing tip. In some embodiments, the high-throughput liquid handler comprises a liquid handling system, such as a pipetting robot. In some embodiments, the pipetting robot can be set to drive fluid at a prescribed pressure, e.g., about 1 to about 20 psi, including about any of 5 psi, 6 psi, 7 psi, 8 psi, 9 psi, 10 psi, 11 psi, 12 psi, 13 psi, 14 psi, or 15 psi.

In some embodiments, provided is a high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir using tubing, the filter comprising a plurality of pores therethrough for perturbing cell membranes of the plurality of cells; a fluid controller configured to control the flow of the liquid carrier from the cell reservoir through the filter to produce the mechanically perturbed cells; a collection well to collect the mechanically perturbed cells in the liquid carrier, and a distributor comprising one or more dispensing tips (e.g., including single or multichannel configurations) for dispensing the liquid carrier comprising the mechanically perturbed cells into two or more samples (e.g., wells in a multi-welled sample plate), wherein the high-throughput system is configured such that the liquid carrier travels from the cell reservoir through the filter then to the collection well prior to the liquid carrier being distributed to the two more samples by way of the distributor. In some embodiments, the tubing has an inner diameter (ID) of about 0.1 mm to about 5 mm, such as about any of 0.5 mm, 0.6 mm, 0.7 mm, 0.8 mm, 0.9 mm, 1 mm, 1.25 mm, 1.5 mm, 1.75 mm, 2 mm, 2.25 mm, 2.5 mm, 2.75 mm, or 3 mm. In some embodiments, the tubing has an inner diameter (ID) and a length such that the pressure drop from the end of the tubing closest to a cell reservoir to the end of the tubing closest to the filter is about 20 psi or less, such as about any of 19 psi or less, 18 psi or less, 17 psi or less, 16 psi or less, 15 psi or less, 14 psi or less, 13 psi or less, 12 psi or less, 11 psi or less, 10 psi or less, 9 psi or less, 8 psi or less, 7 psi or less, 6 psi or less, or 5 psi or less. In some embodiments, the pressure at the filter is about 15 psi or less, such as about any of 14 psi or less, 13 psi or less, 12 psi or less, 11 psi or less, 10 psi or less, 9 psi or less, 8 psi or less, 7 psi or less, 6 psi or less, 5 psi or less, 4 psi or less, 3 psi or less, 2 psi or less, or 1 psi or less. In some embodiments, the high-throughput liquid handler comprises an IR sensor configured to detect the distributor, or a component thereof, in proximity to the collection well. In some embodiments, the high-throughput liquid handler comprises an IR sensor configured to detect the distributor, or a component thereof, in proximity to the collection well such that the liquid carrier comprising cell is processed through the filter to delivery mechanically perturbed cells to the collection well (such as to minimize residency time as described herein).

### III. Methods

Provided herein, in certain aspects, are methods of producing mechanically perturbed cells and methods of using high-throughput systems described herein.

In some aspects, provided herein is a method of performing a cellular assay, the method comprising preparing a plurality of cell-containing samples using a high-throughput liquid handler described herein, wherein the plurality of cell-containing samples comprises two or more samples separately having two or more populations of a cell comprising one or more different payloads delivered thereto; and performing the cellular assay on plurality of cell-containing samples. In some embodiments, the method comprises preparing at least about 100 cell-containing samples, such as at least about 100 to about 10,000. In some embodiments, the plurality of cell-containing samples is prepared in 30 minutes or less, such as about any of 25 minutes or less, 20 minutes or less, 15 minutes or less, 10 minutes or less, 9 minutes or less, 8 minutes or less, 7 minutes or less, 6 minutes or less, 5 minutes or less, 4 minutes or less, 3 minutes or less, 2 minutes or less, or 1 minute or less.

In some aspects, provided herein is a method of performing a cellular assay, the method comprising preparing a plurality of cell-containing samples comprising: passing a plurality of cells through a filter comprising a plurality of pores therethrough for perturbing cell membranes of the plurality of cells; distributing the mechanically perturbed cells to the plurality of cell-containing samples, wherein the plurality of cell-containing samples comprises two or more samples separately having two or more populations of a cell comprising one or more different payloads delivered thereto; and performing the cellular assay on plurality of cell-containing samples.

Cellular assays are well known in the art and cover a diverse array of processes and read-outs, e.g., Nierode et al., J Microbiol Biotechnol, 26, 2016, which is hereby incorporated herein by reference in its entirety. In some embodiments, the cellular assay is selected from the group consisting of a candidate, or combination, compound screen, a cell interaction assay, a cell proliferation or viability assay, a cytotoxicity assay, a metabolic assay, an activity assay, and a polypeptide and/or nucleic acid assay. In some embodiments, the cellular assay is an intracellular binding assay. In some embodiments, the cellular assay is a gene expression and/or gene function assay. In some embodiments, the cellular assay is a binding assay or surrogate thereof, such as a small molecule binding assay.

In some embodiments, the preparing comprises subjecting the cell, in the presence of one or more payloads, to mechanical deformation to effect cellular delivery. In some embodiments, the preparing comprises mechanically deforming the cell and then admixing the mechanically deformed cell to one or more payloads to effect cellular delivery.

In some embodiments, the performing the cellular assay comprises assessing a characteristic of the cell or a component thereof. In some embodiments, the performing the cellular assay comprises imaging at least a portion of the plurality of cell-containing aliquots, assessing a read-out selected from the group consisting of absorbance, fluorescence, luminescence, colorimetric, and radioactivity, performing FACS, or a genetic read-out, such as via a sequence technique, such as PCR or next-generation sequencing. In some embodiments, the cellular assay comprises RNA seq.

Also included are additional method steps relevant to the use of the methods and systems herein. For example, in some embodiments, the method further comprises obtaining a cell or cells, such as from an individual. In some embodiments, the method further comprises obtaining a cell suspension comprising a cell or cells. In some embodiments, the method further comprises preparing a mechanical deformation technique, or component thereof, such as a material having pores therethrough, for a method of delivery as described herein (such as washing said material). In some embodiments, the method further incubating a cell or cells processed with a mechanical deformation such that the cell or cells close the pores made therein. In some embodiments, the method further comprises storing a cell suspension or cells subjected to the delivery methods described herein. In some embodiments, the method further comprises validating delivery of cargo to a cell or cells following performing a delivery method described herein.

### IV. Embodiments

Embodiment 1. A high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples.

Embodiment 2. The high-throughput liquid handler of embodiment 1, wherein the high-throughput liquid handler comprises a single filter comprising the plurality of pores for perturbing cell membranes of the plurality of cells.

Embodiment 3. The high-throughput liquid handler of embodiment 1, wherein the high-throughput liquid handler comprises a second filter comprising a plurality of pores therethrough for perturbing cell membranes of the plurality of cells, and the distributor is configured to dispense the liquid carrier comprising the mechanically perturbed cells from the second filter to two or more samples.

Embodiment 4. The high-throughput liquid handler of any one of embodiments 1-3, wherein the filter comprises at least about 1,000 pores.

Embodiment 5. The high-throughput liquid handler of any one of embodiments 1-4, wherein the filter is configured to process at least about 1 million cells.

Embodiment 6. The high-throughput liquid handler of embodiment 5, wherein the filter is configured to process at least about 5 million cells in more than about 50 μL a liquid carrier.

Embodiment 7. The high-throughput liquid handler of embodiment 5 or 6, wherein the filter is configured to process at least about 5 million cells in about 30 minutes or less.

Embodiment 8. The high-throughput liquid handler of any one of embodiments 1-7, wherein the distributed, mechanically perturbed cells, when subjected to a condition to effect delivery of a payload to the two or more samples, exhibit about 20% or less change in cell viability as compared to the average.

Embodiment 9. The high-throughput liquid handler of any one of embodiments 1-8, wherein the high-throughput liquid handler is configured such that the residency time of the liquid carrier, as measured as the time between exiting the filter and exiting the one or more dispensing tips of the distributor to the sample, is about 2 minutes or less.

Embodiment 10. The high-throughput liquid handler of embodiment 9, wherein the residency time is about 30 seconds or less.

Embodiment 11. The high-throughput liquid handler of embodiment 9 or 10, wherein the residency time is about 0.01 seconds to about 5 seconds.

Embodiment 12. The high-throughput liquid handler of any one of embodiments 1-11, wherein the filter is a silicon filter or a polymer filter.

Embodiment 13. The high-throughput liquid handler of any one of embodiments 1-12, wherein the filter is a silicon filter comprising silicon, silicon oxide, silicon nitride, and/or silicon carbide.

Embodiment 14. The high-throughput liquid handler of any one of embodiments 1-12, wherein the filter comprises a polymer substrate comprising polycarbonate, polyester, polyethersulfone, polyacrylonitrile (PAN), polypropylene, PVDF, polyimide, and/or polytetrafluorethylene.

Embodiment 15. The high-throughput liquid handler of any one of embodiments 1-14, wherein the filter is coated with gold, silver, or platinum.

Embodiment 16. The high-throughput liquid handler of any one of embodiments 1-15, wherein the filter is doped with boron, gallium, or phosphorous.

Embodiment 17. The high-throughput liquid handler of any one of embodiments 1-16, wherein the filter is configured to withstand a pressure of at least 13 psi.

Embodiment 18. The high-throughput liquid handler of any one of embodiments 1-17, wherein each pore of the plurality of pores comprises a cross-section with a dimension of between 2 μm and 20 μm.

Embodiment 19. The high-throughput liquid handler of embodiment 18, wherein a pitch between each pore of the plurality of pores is between 0.5:1 and 100:1 relative to the largest cross-section dimension of the pore.

Embodiment 20. The high-throughput liquid handler of any one of embodiments 1-19, wherein the filter comprises a filtering surface and a support structure disposed on a side of the filtering surface, the plurality of pores extending through the filtering surface.

Embodiment 21. The high-throughput liquid handler of embodiment 20, wherein the support structure covers at least 1% of the filtering surface.

Embodiment 22. The high-throughput liquid handler of any one of embodiments 20 or 21, wherein the support structure comprises a plurality of supporting members disposed on the filtering surface and forming at least one cross shape or a plurality of stripes.

Embodiment 23. The high-throughput liquid handler of any one of embodiments 20-22, wherein a thickness of the support structure is between 20 μm and 1 mm.

Embodiment 24. The high-throughput liquid handler of any one of embodiments 20-23, wherein a thickness of the filtering surface is between 0.1 μm and 100 μm.

Embodiment 25. The high-throughput liquid handler of any one of embodiments 20-24, wherein a width of the filtering surface is between 1 mm and 20 mm.

Embodiment 26. The high-throughput liquid handler of any one of embodiments 20-25, wherein the filter comprises an oxide layer disposed on the filtering surface between the filtering surface and the support structure.

Embodiment 27. The high-throughput liquid handler of any one of embodiments 1-26, wherein the fluid controller, in controlling the flow of the liquid carrier from the cell reservoir through the filter, is configured to create a pressure of at least about 1 psi as measured in the liquid carrier on the upstream adjacent side of the filter.

Embodiment 28. The high-throughput liquid handler of any one of embodiments 1-27, wherein the fluid controller comprises a syringe pump or a peristaltic pump.

Embodiment 29. The high-throughput liquid handler of any one of embodiments 1-28, wherein the syringe pump moves the liquid carrier at a rate of about 0.5 mL/minute to about 60 mL/minute.

Embodiment 30. The high-throughput liquid handler of any one of embodiments 1-29, wherein the fluid controller comprises a solenoid, and wherein the cell reservoir is configured to apply a force to the liquid carrier such that the liquid carrier moves towards the solenoid and the filter.

Embodiment 31. The high-throughput liquid handler of embodiment 30, wherein the solenoid is configured to complete a closed-to-open-to-closed cycle in about 1 millisecond or more.

Embodiment 32. The high-throughput liquid handler of embodiment 30 or 31, wherein the solenoid is configured to create sample volumes of about 1 μL to about 500 μL.

Embodiment 33. The high-throughput liquid handler of any one of embodiments 1-32, wherein the solenoid is configured to create a sample volume of 25 μL or less.

Embodiment 34. The high-throughput liquid handler of any one of embodiments 30-33, wherein the solenoid is an electromechanical solenoid.

Embodiment 35. The high-throughput liquid handler of any one of embodiments 1-34, wherein the fluid controller, in controlling the flow of the liquid carrier from the cell reservoir through the filter, is configured to pass the liquid carrier through the filter at a volumetric flow rate of between 0.5 mL/min per mm² porous surface area and 500 mL/min per mm² porous surface area.

Embodiment 36. The high-throughput liquid handler of any one of embodiments 1-35, wherein the one or more dispensing tips of the distributor are pipette tips.

Embodiment 37. The high-throughput liquid handler of any one of embodiments 1-36, wherein the distributor comprises a single dispensing tip.

Embodiment 38. The high-throughput liquid handler of any one of embodiments 1-36, wherein the distributor comprises a gantry.

Embodiment 39. The high-throughput liquid handler of any one of embodiments 1-38, wherein the one or more dispensing tips of the distributor are configured for non-contact liquid dispensing.

Embodiment 40. The high-throughput liquid handler of any one of embodiments 1-37 or 39, wherein the filter, the fluid controller, and the distributor are configured as an end-effector comprising a solenoid positioned upstream and in fluidic communication with a filter positioned upstream and in fluidic communication with the one or more dispensing tips.

Embodiment 41. The high-throughput liquid handler of embodiment 40, wherein the end-effector is a removable and replaceable cartridge.

Embodiment 42. The high-throughput liquid handler of any one of embodiments 1-41, wherein the reservoir is pressurized such that a force is applied to the liquid carrier therein in a manner that liquid carrier moves towards the filter.

Embodiment 43. The high-throughput liquid handler of embodiment 42, wherein the pressurization of the reservoir is a positive pressure.

Embodiment 44. The high-throughput liquid handler of embodiment 42 or 43, wherein the reservoir is pressurized with a gas.

Embodiment 45. The high-throughput liquid handler of embodiment 44, wherein the gas is air, CO₂, an inert gas, or any mixture thereof.

Embodiment 46. The high-throughput liquid handler of embodiment 42 or 43, wherein the reservoir is pressurized with a pump.

Embodiment 47. The high-throughput liquid handler of embodiment 46, wherein the pump is a peristaltic pump.

Embodiment 48. The high-throughput liquid handler of embodiment 42, wherein the pressurization of the reservoir is a negative pressure.

Embodiment 49. The high-throughput liquid handler of any one of embodiments 1-48, further comprising a collection well configured to collect the liquid carrier comprising the mechanically perturbed cells from the filter prior to the distributor dispensing the liquid carrier comprising the mechanically perturbed cells from the collection well into the two or more samples.

Embodiment 50. A high-throughput liquid handler configured for distribution of mechanically perturbed cells, the high-throughput liquid handler comprising: a syringe comprising an outlet and a cell reservoir configured to hold a liquid carrier comprising a plurality of cells; a filter placed in fluidic communication with the cell reservoir of the syringe via the outlet, the filter comprising a plurality of pores therethrough for perturbing cell membranes of the plurality of cells; a fluid controller comprising a syringe pump configured to control the flow of the liquid carrier from the cell reservoir through the filter to produce the mechanically perturbed cells; a collection well situated to collect the liquid carrier comprising the mechanically perturbed cells from the filter; and a distributor comprising one or more dispensing tips for dispensing the liquid carrier comprising the mechanically perturbed cells from the collection well into two or more samples.

Embodiment 51. A high-throughput liquid handler configured for distribution of mechanically perturbed cells, the high-throughput liquid handler comprising: a syringe comprising an outlet and a filter placed in the fluidic path of the outlet, the filter comprising a plurality of pores therethrough for perturbing cell membranes of the plurality of cells; a cell reservoir configured to hold a liquid carrier comprising a plurality of cells; a fluid controller comprising a syringe pump configured to control the flow of the liquid carrier through the filter such that the liquid carrier comprising the plurality of cells is aspirated from the cell reservoir through the filter and then dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples.

Embodiment 52. The high-throughput liquid handler of embodiment 50 or 51, wherein the distributor comprising the one or more dispensing tips is a gantry.

Embodiment 53. A high-throughput liquid handler configured for distribution of mechanically perturbed cells, the high-throughput liquid handler comprising: a cell reservoir configured to hold a liquid carrier comprising a plurality of cells; a filter configured to be placed in fluidic communication with the cell reservoir, the filter comprising a plurality of pores therethrough for perturbing cell membranes of the plurality of cells; a fluid controller comprising a solenoid configured to control the flow of the liquid carrier from the cell reservoir through the filter to produce the mechanically perturbed cells; and a distributor comprising one or more dispensing tips for dispensing the liquid carrier comprising the mechanically perturbed cells into two or more samples.

Embodiment 54. The high-throughput liquid handler of embodiment 53, wherein the reservoir is pressurized such that a force is applied to the liquid carrier therein in a manner that liquid carrier moves towards the filter.

Embodiment 55. The high-throughput liquid handler of embodiment 54, wherein the pressurization of the reservoir is a positive pressure.

Embodiment 56. The high-throughput liquid handler of embodiment 54 or 55, wherein the reservoir is pressurized with a gas.

Embodiment 57. The high-throughput liquid handler of embodiment 56, wherein the gas is air, CO₂, an inert gas, or any mixture thereof.

Embodiment 58. The high-throughput liquid handler of embodiment 54 or 55, wherein the reservoir is pressurized with a pump.

Embodiment 59. The high-throughput liquid handler of embodiment 58, wherein the pump is a peristaltic pump.

Embodiment 60. The high-throughput liquid handler of any one of embodiments 53-59, wherein the filter, the fluid controller, and the distributor are configured as an end-effector comprising a solenoid positioned upstream and in fluidic communication with a filter positioned upstream and in fluidic communication with the one or more dispensing tips.

Embodiment 61. The high-throughput liquid handler of embodiment 60, wherein the end-effector is a removable and replaceable cartridge.

Embodiment 62. A high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells is dispensed through the filter to a collection well situated to collect the liquid carrier comprising the mechanically perturbed cells; and a distributor comprising one or more dispensing tips for dispensing the liquid carrier comprising the mechanically perturbed cells from the collection well into two or more samples.

Embodiment 63. The high-throughput liquid handler of embodiment 62, wherein the distributor comprising the one or more dispensing tips is a gantry.

Embodiment 64. A high-throughput liquid handler configured for distribution of mechanically perturbed cells, the high-throughput liquid handler comprising: a syringe comprising an outlet, a filter placed in the fluidic path of the outlet, the filter comprising a plurality of pores therethrough for perturbing cell membranes of the plurality of cells, and a cell reservoir configured to hold a liquid carrier comprising a plurality of cells; a fluid controller comprising a syringe pump configured to control the flow of the liquid carrier through the filter such that the liquid carrier comprising the plurality of cells flows through the filter to produce mechanically perturbed cells; and a distributor comprising one or more dispensing tips for dispensing the liquid carrier comprising the mechanically perturbed cells from the syringe into two or more samples.

Embodiment 65. The high-throughput liquid handler of embodiment 64, wherein the distributor comprising the one or more dispensing tips is a gantry.

Embodiment 66. The high-throughput liquid handler of any one of embodiments 1-65, further comprising a sample holder configured to secure a multi-well plate having 2 or more wells to receive the two or more samples.

Embodiment 67. The high-throughput liquid handler of embodiment 66, wherein the multi-well plate comprises 96 wells.

Embodiment 68. The high-throughput liquid handler of embodiment 66, wherein the multi-well plate comprises 384 wells.

Embodiment 69. The high-throughput liquid handler of embodiment 66, wherein the multi-well plate comprises 1536 wells.

Embodiment 70. The high-throughput liquid handler of any one of embodiments 66-69, wherein the sample holder comprises an x- and y-plane positioner.

Embodiment 71. The high-throughput liquid handler of any one of embodiments 66-70, wherein the sample holder comprises an x-, y-, and z-plane positioner.

Embodiment 72. The high-throughput liquid handler of any one of embodiments 1-71, further comprising one or more robotic arms, the one or more robotic arms separately connected, directly or indirectly, to one or more of the filter, the fluid controller, or the distributor.

Embodiment 73. The high-throughput liquid handler of embodiment 72, wherein the robotic arm comprises an x- and y-plane positioner.

Embodiment 74. The high-throughput liquid handler of embodiment 72 or 73, wherein the robotic arm comprises an x-, y-, and z-plane positioner.

Embodiment 75. The high-throughput liquid handler of any one of embodiments 1-74, wherein the cell reservoir is configured to be placed in fluidic communication with the filter using tubing.

Embodiment 76. The high-throughput liquid handler of any one of embodiments 1-75, wherein the high-throughput liquid handler is communicatively couplable to a control system to control one or more operating parameters of the high-throughput liquid handler.

Embodiment 77. The high-throughput liquid handler of any one of embodiments 1-76, comprising a control system communicatively coupled to the high-throughput liquid handler to control one or more operating parameters of the high-throughput liquid handler.

Embodiment 78. The high-throughput liquid handler of embodiment 76 or 77, wherein the control system comprises: one or more processors; and memory storing one or more programs, the one or more programs configured to be executed by the one or more processors, the one or more programs including instructions for at least one of: controlling the flow the liquid carrier; opening a solenoid; closing the solenoid; controlling fluid volume in a syringe; positioning one or more robotic arms.

Embodiment 79. The high-throughput liquid handler of any one of embodiments 1-78, further comprising the liquid carrier.

Embodiment 80. The high-throughput liquid handler of embodiment 79, wherein the liquid carrier comprises a buffer.

Embodiment 81. The high-throughput liquid handler of embodiment 79 or 80, wherein the liquid carrier has a volume of about 50 μL to about 100 mL.

Embodiment 82. The high-throughput liquid handler of any one of embodiments 79-81, wherein the liquid carrier comprises about 100,000 to about 500 million cells per mL.

Embodiment 83. The high-throughput liquid handler of any one of embodiments 79-82, wherein the liquid carrier comprises one or more payloads to be delivered to the mechanically perturbed cells.

Embodiment 84. The high-throughput liquid handler of any one of embodiments 1-83, wherein the two or more distributed samples of mechanically perturbed cells are each distributed to a sample well, wherein one or more payloads to be delivered to the mechanically perturbed cells are present in the sample well when the mechanically perturbed cells are distributed thereto.

Embodiment 85. The high-throughput liquid handler of any one of 83 or 84, wherein the one or more payloads are individually selected from the group consisting of a polypeptide, a nucleic acid, a small molecule compound, and a polymer.

Embodiment 86. The high-throughput liquid handler of any one of embodiments 1-85, further comprising the plurality of cells.

Embodiment 87. The high-throughput liquid handler of embodiment 86, wherein the plurality of cells comprises a somatic cell, a stem cell, an immortalized cell, or a derivative thereof.

Embodiment 88. The high-throughput liquid handler of embodiment 86 or 87, wherein the plurality of cells comprises a PBMC cell, or a derivative thereof.

Embodiment 89. The high-throughput liquid handler of any one of embodiments 86-88, wherein the plurality of cells comprises an immune cell or a derivative thereof.

Embodiment 90. The high-throughput liquid handler of embodiment 89, wherein the immune cell is a T cell, natural killer (NK) cell, monocyte, B cell, or dendritic cell.

Embodiment 91. The high-throughput liquid handler of embodiment 87, wherein the cell is a stem cell or a derivative thereof.

Embodiment 92. The high-throughput liquid handler of embodiment 91, wherein the cell is an induced pluripotent stem cell, a hematopoietic cell, or a mesenchymal cell.

Embodiment 93. The high-throughput liquid handler of embodiment 87, wherein the immortalized cell is a cancer cell or a derivative thereof.

Embodiment 94. The high-throughput liquid handler of any one of embodiments 86-93, wherein the cell is obtained, or derived, from an individual.

Embodiment 95. The high-throughput liquid handler of embodiment 94, wherein the individual is a human.

Embodiment 96. The high-throughput liquid handler of any one of embodiments 1-95, further comprising a multi-well plate configured to receive the two or more samples.

Embodiment 97. The high-throughput liquid handler of embodiment 96, wherein the high-throughput liquid handler is configured to dispense a sample comprising the mechanically perturbed cells to 96 individual wells within about 30 seconds.

Embodiment 98. The high-throughput liquid handler of embodiment 96, wherein the high-throughput liquid handler is configured to dispense a sample comprising the mechanically perturbed cells to 384 individual wells within about 30 seconds.

Embodiment 99. The high-throughput liquid handler of embodiment 96, wherein the high-throughput liquid handler is configured to dispense a sample comprising the mechanically perturbed cells to 1536 individual wells within about 30 seconds.

Embodiment 100. A cartridge for use in any one of the high-throughput liquid handlers of embodiments 1-99, wherein the cartridge comprises a filter comprising a plurality of pores therethrough for perturbing cell membranes of a plurality of cells, and a distributor comprising one or more dispensing tips for dispensing the mechanically perturbed cells into two or more samples.

Embodiment 101. A cartridge for use in any one of the high-throughput liquid handlers of embodiments 1-99, wherein the cartridge comprises a fluid controller comprising a solenoid configured to control the flow of a liquid carrier through a filter to produce mechanically perturbed cells, the filter comprising a plurality of pores therethrough for perturbing cell membranes of a plurality of cells, and a distributor comprising one or more dispensing tips for dispensing the mechanically perturbed cells into two or more samples.

Embodiment 102. A method of performing a cellular assay, the method comprising preparing a plurality of cell-containing samples using the high-throughput liquid handler of any one of embodiments 1-99, wherein the plurality of cell-containing samples comprises two or more samples separately having two or more populations of a cell comprising one or more different payloads delivered thereto; and performing the cellular assay on plurality of cell-containing samples.

Embodiment 103. The method of embodiment 102, wherein the method comprises preparing at least about 100 cell-containing samples.

Embodiment 104. The method of embodiments 102 or 103, wherein the plurality of cell-containing samples is prepared in 30 minutes or less.

Embodiment 105. A method of performing a cellular assay, the method comprising preparing a plurality of cell-containing samples comprising: passing a plurality of cells through a filter comprising a plurality of pores therethrough for perturbing cell membranes of the plurality of cells; distributing the mechanically perturbed cells to the plurality of cell-containing samples, wherein the plurality of cell-containing samples comprises two or more samples separately having two or more populations of a cell comprising one or more different payloads delivered thereto; and performing the cellular assay on plurality of cell-containing samples.

Embodiment 106. The method of any one of embodiments 102-105, wherein the cellular assay is selected from the group consisting of a candidate, or combination, compound screen, a cell interaction assay, a cell proliferation or viability assay, a cytotoxicity assay, a metabolic assay, an activity assay, and a polypeptide and/or nucleic acid assay.

Embodiment 107. The method of any one of embodiments 102-106, wherein the preparing comprises subjecting the cell, in the presence of one or more payloads, to mechanical deformation to effect cellular delivery.

Embodiment 108. The method of any one of embodiments 102-107, wherein the preparing comprises mechanically deforming the cell and then admixing the mechanically deformed cell to one or more payloads to effect cellular delivery.

Embodiment 109. The method of any one of embodiments 102-108, wherein the performing the cellular assay comprises assessing a characteristic of the cell or a component thereof.

Embodiment 110. The method of any one of embodiments 102-109, wherein the performing the cellular assay comprises imaging at least a portion of the plurality of cell-containing aliquots, assessing a read-out selected from the group consisting of absorbance, fluorescence, luminescence, colorimetric, and radioactivity, performing FACS, or a genetic read-out.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

This example demonstrates a high-throughput system and method for delivery of payloads to induced pluripotent stem cells (iPSCs) using various residency times between mechanical perturbation of the iPSCs and introduction of the payload.

iPSCs were lifted into a single cell suspension using Accutase and resuspended at 10 million cells/mL in mTeSR basal media. To prepare the payload, a mixture of AF647 10 kDa dextran and Cascade blue 3 kDa dextran were diluted at 0.1 mg/mL and prepared in mTeSR basal media. Dextran is a model payload useful for evaluating the delivery of various payloads, e.g., polypeptides and nucleic acids. 12.5 μL of the payload mixture was distributed into wells of a 96-well V-bottom tissue culture plate. 110 μL of the cell suspension was loaded into a cartridge containing a filter with defined pore sizes (10 μm). The cartridge was connected to a system for passing the cells through the filter at 3 psi. The mechanically perturbed cells were collected in a collection well. 12.5 μL aliquots of cells from the collection well were added to wells of 96-well plate at 3-4 second intervals according to desired residency times to be evaluated (the time between mechanically perturbing the cells to addition to the well with the payload). Replicates evaluating different timing of addition of cells to payload were completed. The 96-well plate was subsequently spun down in a centrifuge, and the cells were washed 3× with a buffer containing PBS, 2% FBS, and 0.2% EDTA before reading out dextran delivery using a flow cytometer.

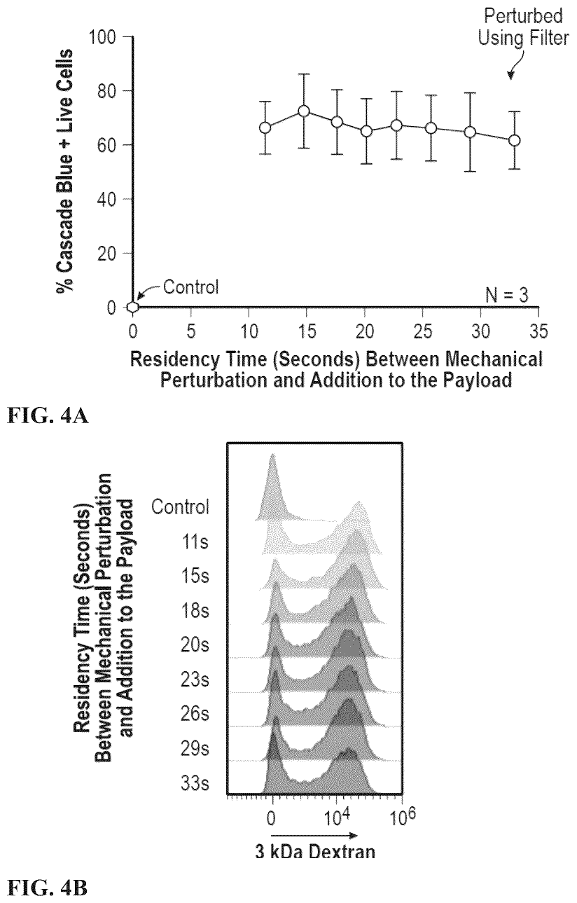

The results from the flow cytometry analysis are provided in FIGS. 4A and 4B. As shown in FIGS. 4A and 4B, dextran was delivered to iPSCs across various residency times.

### Example 2

This example demonstrates a high-throughput system and method for delivering payloads to induced pluripotent stem cells (iPSCs) using various residency times between mechanical perturbation of the iPSCs and introduction of the payload.

iPSCs were lifted into a single cell suspension using Accutase and resuspended at 20 million cells/mL in mTeSR basal media. To prepare the payload, a mixture of AF647 10 kDa dextran and Cascade blue 3 kDa dextran were diluted at 0.1 mg/mL and prepared in mTeSR basal media. Dextran is a model payload useful for evaluating the delivery of various payloads, e.g., polypeptides and nucleic acids. 12.5 μL of the payload mixture was distributed into wells of a 96-well V-bottom tissue culture plate. 110 μL of the cell suspension was loaded into a cartridge containing a filter with defined pore sizes (10 μm). The cartridge was connected to a system for passing the cells through the filter at 3 psi. The mechanically perturbed cells were collected in collection well. 12.5 μL aliquots of cells from the collection well were added to wells of 96-well plate at 15 second intervals according to desired residency times to be evaluated (the time between mechanically perturbing the cells to addition to the well with the payload). Replicates evaluating different timing of addition of cells to payload were completed. The 96-well plate was subsequently spun down in a centrifuge, and the cells were washed 3× with a buffer containing PBS, 2% FBS, and 0.2% EDTA before reading out dextran delivery using a flow cytometer. Cell viability was also assessed following delivery to assess for live cells.

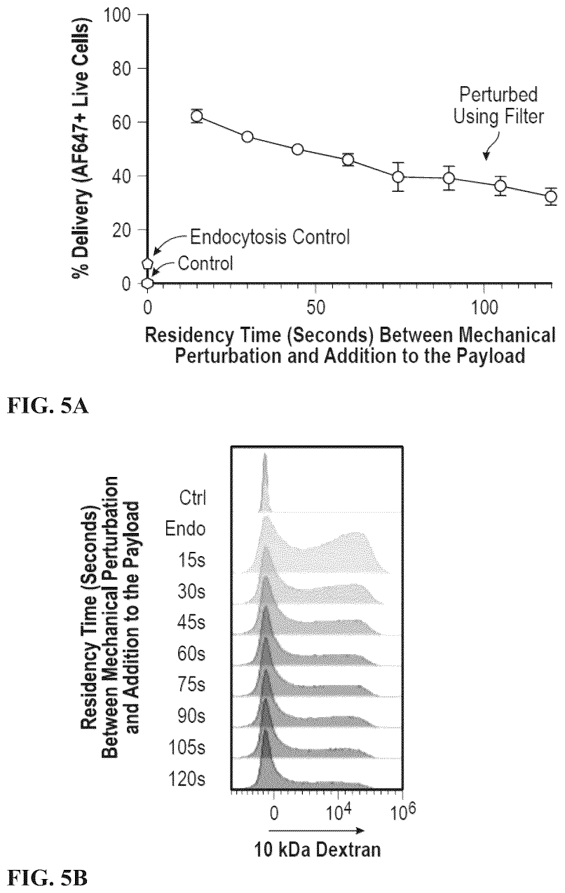

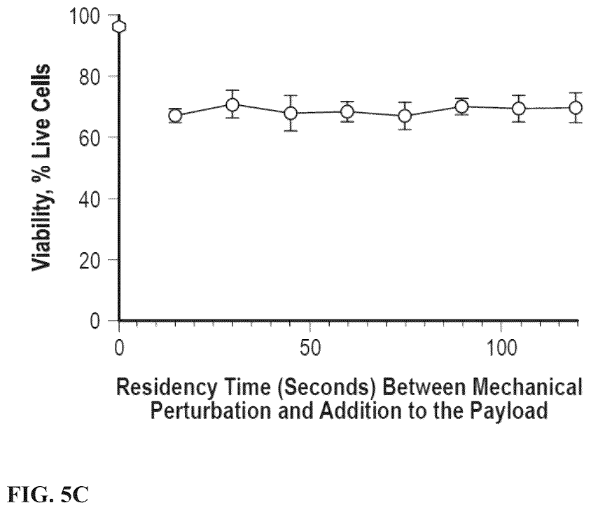

The results from the flow cytometry analysis are provided in FIGS. 5A and 5B. As shown in FIGS. 5A and 5B, dextran was delivered to iPSCs across various residency times. Intracellular delivery of dextran was observed to decrease for certain longer residency times, e.g., 105 seconds. Cell viability was not impacted by the mechanical perturbation of the cells across all residency times evaluated (FIG. 5C).

### Example 3

This example demonstrates a high-throughput system and method for delivering payloads to HeLa cells using various residency times between mechanical perturbation of the HeLa cells and introduction of the payload.

HeLa cells were lifted into a single cell suspension using 0.25% Trypsin-EDTA and resuspended at 20 million cells/mL in Opti-MEM media. To prepare the payload, a mixture of AF647 10 kDa dextran, Cascade blue 3 kDa dextran, and AF488 3 kDa dextran were diluted at 0.1 mg/mL and prepared in Opti-MEM media. Dextran is a model payload useful for evaluating the delivery of various payloads, e.g., polypeptides and nucleic acids. 12.5 μL of the payload mixture was distributed into wells of a 96-well V-bottom tissue culture plate. 110 μL of cell suspension was loaded into a cartridge containing a filter with defined pore sizes (three sizes evaluated: 10 μm, 11 μm, and 12 μm). The cartridge was connected to a system for passing the cells through the filter at 7 psi. Cells were collected in a collection well. 12.5 μL aliquots of cells from the collection well were added to wells of 96-well plate at 5 second intervals according to desired residency times to be evaluated (the time between mechanically perturbing the cells to addition to the well with the payload). At least after 30 seconds following addition of cells to payload, complete DMEM+GlutaMAX media was added to the cell and payload mixture. The 96-well plate was subsequently spun down in a centrifuge, and the cells were washed 3× with a buffer containing PBS, 2% FBS, and 0.2% EDTA before reading out dextran delivery using a flow cytometer.

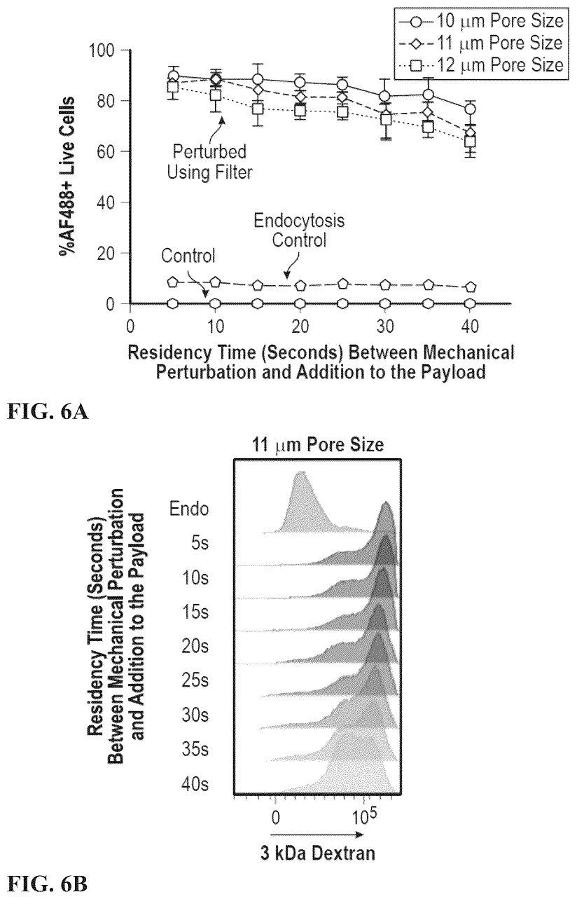

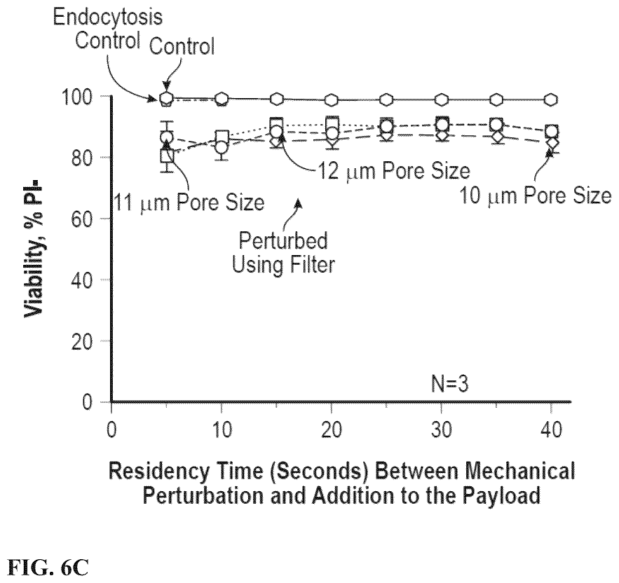

The results from the flow cytometry analysis are provided in FIGS. 6A and 6B. As shown in FIGS. 6A and 6B, dextran was delivered to HeLa cells across various residency times and filter pore sizes. Cell viability was not impacted by the mechanical perturbation of the cells across all residency times and filter pore sizes evaluated (FIG. 6C).

### Example 4

This example demonstrates a high-throughput system and method for delivering payloads to activated T cells using various residency times between mechanical perturbation of the T cells and introduction of the payload.

T cells were activated for 6 days and activation beads were removed on day 3 (diameter as 10.8 μm). Activated T cells were suspended at 40 million cells/mL in Opti-MEM. To prepare the payload, a mixture of AF647 10 kDa dextran and Cascade blue 3 kDa dextran were diluted at 0.1 mg/mL and prepared in Opti-MEM media. Dextran is a model payload useful for evaluating the delivery of various payloads, e.g., polypeptides and nucleic acids. 30 μL of the payload mixture was distributed into wells of a 96-well V-bottom tissue culture plate. 300 μL of cell suspension was loaded into cartridge containing a filter with a defined pore size (7 μm). The cartridge was connected to a system for passing the cells through the filter at 9 psi. Cells were collected in a collection well. 30 μL aliquots of cells from the collection well were added to wells of the 96-well plate at time increments according to desired residency times to be evaluated (the time between mechanically perturbing the cells to addition to the well with the payload). At least after 30 seconds following addition of cells to payload, 60 μL of activated T Cell media was added to the cell and payload mixture. The 96-well plate was subsequently spun down in a centrifuge, and the cells were washed 3× with a buffer containing PBS, 2% FBS, and 0.2% EDTA before reading out dextran delivery using a flow cytometer.

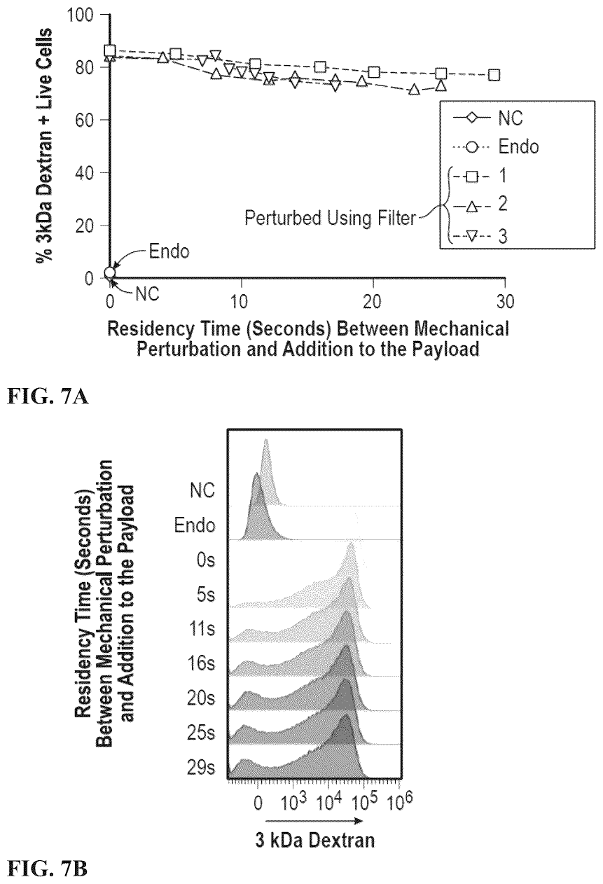

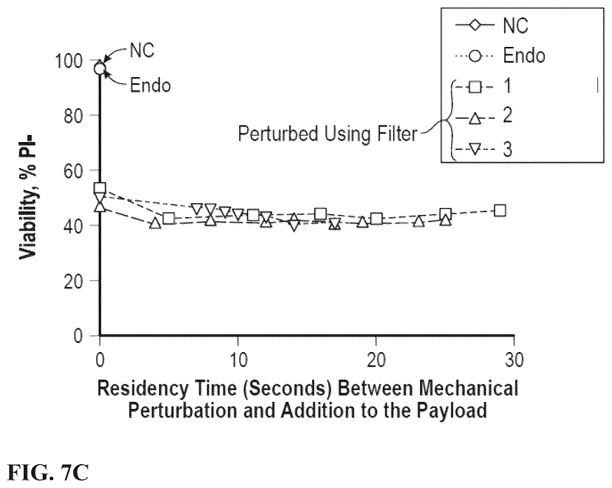

The results from the flow cytometry analysis are provided in FIGS. 7A and 7B. As shown in FIGS. 7A and 7B, dextran was delivered to activated T cells across various residency times. Cell viability was not impacted by the mechanical perturbation of the cells across all residency times evaluated (FIG. 7C).

### Example 5

This example demonstrates a high-throughput system and method for delivering payloads to activated T cells using various residency times between mechanical perturbation of the T cells and introduction of the payload.

T cells were activated for 9 days and activation beads were removed on day 3 (diameter as 8.6 μm). Activated T cells were suspended at 40 million cells/mL in Opti-MEM. To prepare the payload, a mixture of AF647 10 kDa dextran and Cascade blue 3 kDa dextran were diluted at 0.1 mg/mL and prepared in Opti-MEM media. Dextran is a model payload useful for evaluating the delivery of various payloads, e.g., polypeptides and nucleic acids. 20 μL of the payload mixture was distributed into wells of a 96-well V-bottom tissue culture plate. 200 μL of cell suspension was loaded into a cartridge containing a filter with a defined pore size (6 μm). The cartridge was connected to a system for passing the cells through the filter at 10 psi. Cells were collected in collection well. 30 μL aliquots of cells from the collection well were added to wells of the 96-well plate at time increments according to desired residency times to be evaluated (the time between mechanically perturbing the cells to addition to the well with the payload). At least after 30 seconds following addition of cells to payload, 60 μL of activated T Cell media was added to the cell and payload mixture. The 96-well plate was subsequently spun down in a centrifuge, and the cells were washed 3× with a buffer containing PBS, 2% FBS, and 0.2% EDTA before reading out dextran delivery using a flow cytometer.

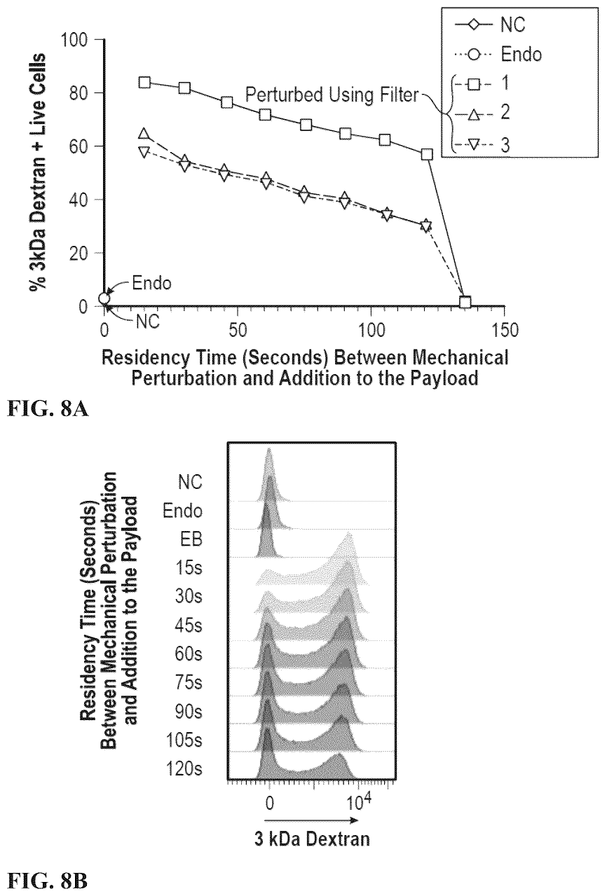

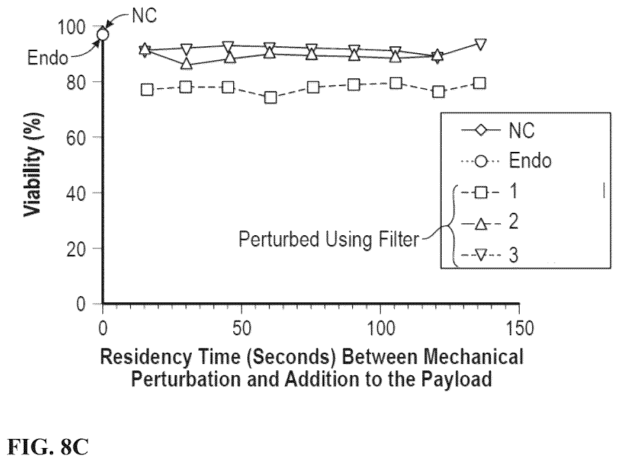

The results from the flow cytometry analysis are provided in FIGS. 8A and 8B. As shown in FIGS. 8A and 8B, dextran was delivered to activated T cells across various residency times, however, a residency time of 135 seconds resulted in insignificant intracellular dextran delivery. Cell viability was not impacted by the mechanical perturbation of the cells across all residency times evaluated (FIG. 8C).

### Example 6

This example demonstrates a high-throughput system and method for delivering payloads to HeLa cells using various residency times between mechanical perturbation of the HeLa cells and introduction of the payload.

HeLa cells were lifted into a single cell suspension using 0.25% Trypsin-EDTA and resuspended at 2 million cells/mL in PBS. Opti-MEM culture media was used for culturing the HeLa cells. To prepare the payload, a Cascade blue 3 kDa dextran was diluted at 0.1 mg/mL and prepared in Opti-MEM media. Dextran is a model payload useful for evaluating the delivery of various payloads, e.g., polypeptides and nucleic acids. 30 μL of the payload mixture was distributed into wells of a 96-well V-bottom tissue culture plate. A cartridge containing a filter with defined pore sizes (10 μm) was connected to a GNF Systems One-Tip Dispenser (Model #1221-9000) via microfluidic tubing. A solenoid valve and dispenser having a dispensing nozzle for plating was connected to the cartridge, such that the order of components from upstream to downstream is filter, solenoid, and dispenser. PBS media was used to prime the tubing and discarded. A conical tube containing the lifted HeLa cells was connected to the pipetting robot via tubing through a cap of the tube. Following the initial priming, HeLa cells in PBS were then primed through the tubing and discarded. The pressure was then set on the pipetting system and L aliquots of the cell suspension was dispensed into each of 24 wells of the 96-well plate in a serpentine pattern (the pipetting system was set to dispense 30 μL with 150% volume scaling per well; pressure of 9 psi and 11 psi were separately tested). The 96-well plate was subsequently spun down in centrifuge, and cells were washed 2× with a buffer containing PBS, 2% FBS, and 0.2% EDTA before reading out dextran delivery using a flow cytometer.

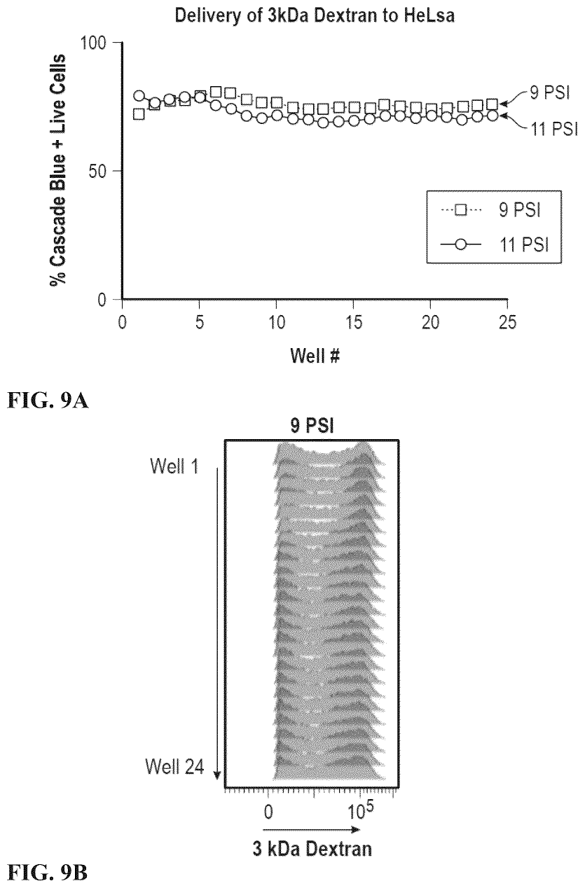

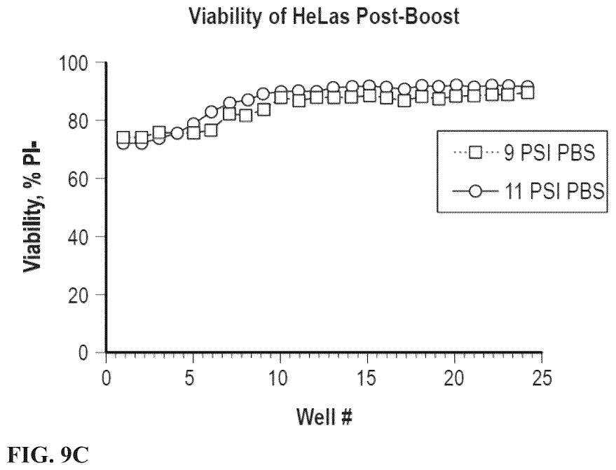

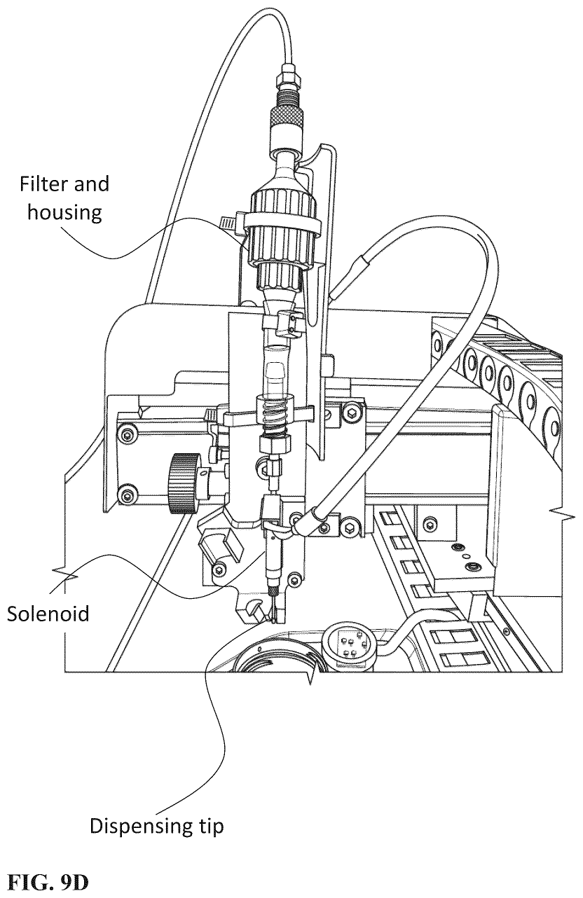

The results from the flow cytometry analysis are provided in FIGS. 9A and 9B. As shown in FIGS. 9A and 9B, dextran was delivered to HeLa cells across various residency times and filter pore sizes. Cell viability was not impacted by the mechanical perturbation of the cells across all residency times and pressures evaluated (FIG. 9C).

### Example 7

This example demonstrates various high-throughput systems and methods for delivering payloads to PBMCs. Characteristics of the tubing inner diameter (ID) size for transporting a cell suspension from a pressurized cell reservoir to a silicon filter where mechanoporation occurs were evaluated. Specifically, the tubing evaluated was configured to carry a cell suspension from a pressurized 50 mL conical tube to a silicon membrane where mechanoporation occurs. PBMCs were isolated by Ficoll separation from a Leukopak, and frozen for storage. On the day of mechanoporation, the PBMCs were thawed and prepared for testing. The cells were suspended in Opti-Mem at 2 million/mL. 3 kDa Cascade blue Dextan was pre-mixed into the cell suspension at a concentration of 100 μg/mL. Two tubing sizes, 0.8 mm and 2.0 mm ID, were tested at pressures of 3, 6, 9, 12, and 15 PSI. The tubing length was 300 mm for both ID sizes. Tests were performed from low to high pressure. A new, 5 micron, 10×10 mm, silicon filter was used after performing the pressure sweep for each tubing size. Filters were loaded into cartridges connected to a high-throughput system for passing the cells through the filter, which contain a 50A Durometer O-ring, with 8.25 mm ID and a 5 mm Cross Section. The cartridge was designed to seal the O-ring with a 32% Compression Ratio. Controls were performed on a research scale system, configured for lower volume liquid carrier processing, such as less than 10 mL, as compared to the high-throughput system.

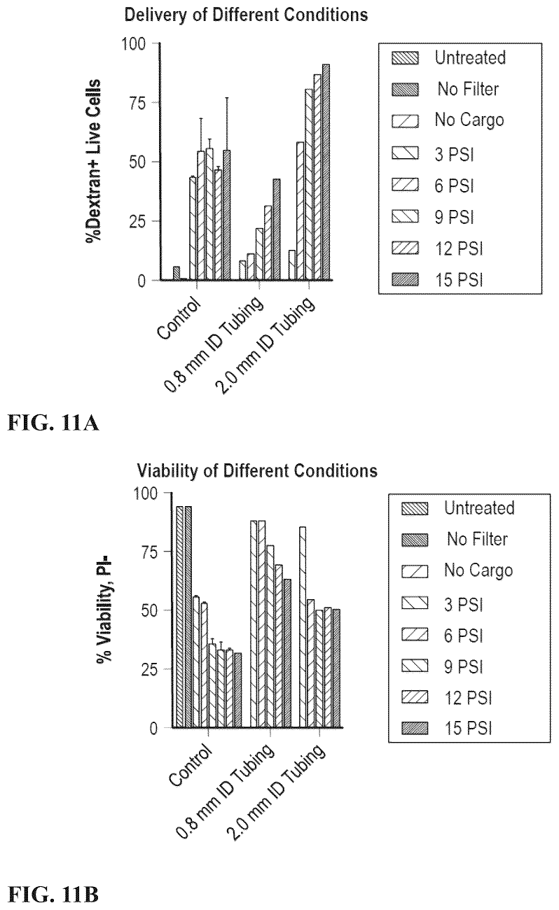

Delivery of 3 kDa dextran was assessed with Flow Cytometry (FIG. 11A). Propidium Iodine stain was used to measure viability on flow cytometry (FIG. 11B). Delivery increased with increasing pressure for both tubing inner diameter sizes. The increase in delivery with increasing pressure was greater when using the 2 mm ID tubing. There is a roughly 4-fold increase in delivery at 6 PSI, and a 3-fold increase at 9 PSI when switching to the 2 mm ID tubing. Cell viability decreased at higher pressures in both tubing setups. The decrease in viability was stronger with the 2.0 mm ID tubing, but leveled off at around 50% viability for 9 PSI and higher. This experiment showed that resistance to flow from small ID tubing can decrease delivery of dextran in PBMCs. Larger tubing reduced the flow resistance, and allowed for high delivery within the 0 to 15 PSI operating range.

### Example 8

This example demonstrates a high-throughput system described herein for intracellular delivery to iPSCs, wherein the system is configured with 2.0 mm inner diameter (ID) tubing connecting a cell reservoir to a silicon filter where mechanoporation occurs. Performance was compared with a control research scale system.

IPSCs were cultured for this experiment in mTESR plus media. They were harvested and re-suspended in mTESR plus media at a concentration of 2 million/mL. 3 kDa cascade blue dextran was pre-mixed with the cells at a final concentration of 100 μg/mL. A 10 micron, 10×10 mm silicon chip was used on the high-throughput system. Filters were loaded into cartridges, which contained a 50A Durometer O-ring, with 8.25 mm ID and a 5 mm Cross Section. The cartridge is designed to seal the O-ring with a 32% Compression Ratio. A 10 micron, 5×5 mm silicon membrane was used on control research scale system.

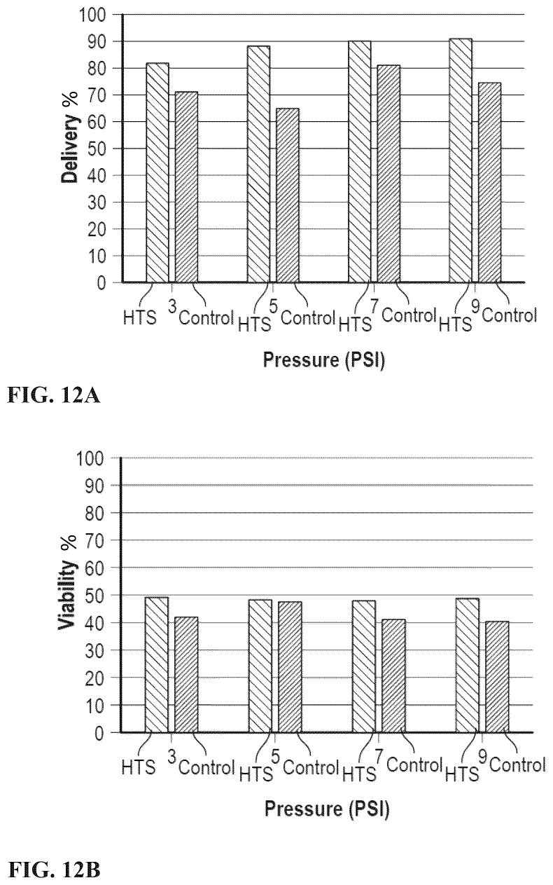

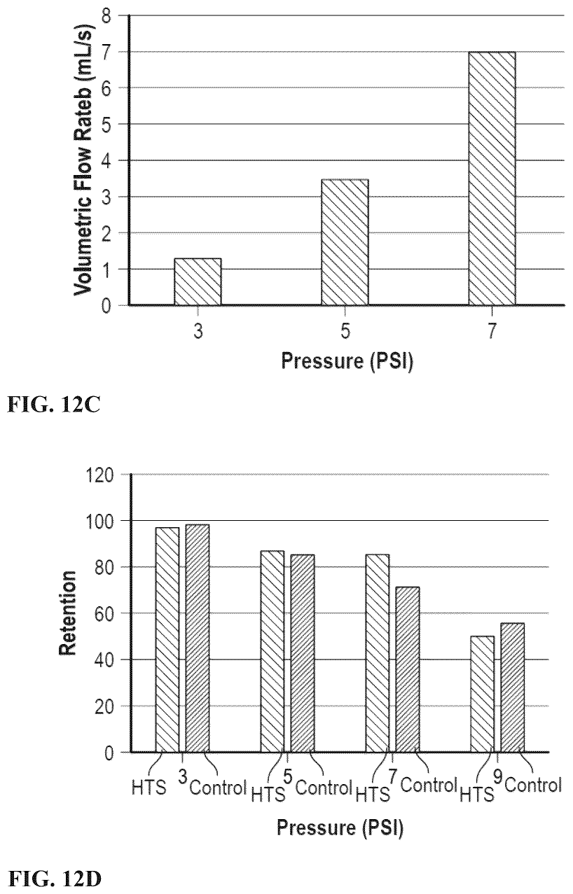

Delivery of 3 kDa dextran was assessed with flow cytometry (FIG. 12A). Propidium Iodine stain was used to measure viability on flow cytometry (FIG. 12B). Delivery increased with increasing pressure on the high-throughput system. Delivery was higher across all conditions on the high-throughput system versus the control research scale system. There was no measurable change in viability on the high-throughput system with varied pressures. The volumetric flow rate of each condition was calculated (FIG. 12C) and it was found that volumetric flow rate increased with increasing pressure. Retention, which is a reflection of the number of cells lost during the mechanoporation process, was calculated by event count on flow cytometry, relative to the endocytosis control (FIG. 12D). This experiment showed that 2.0 mm ID tubing can be used for iPSCs on the high-throughput system and that the high-throughput system provided improved delivery performance.

### Example 9

This example demonstrates use of a high-throughput system described herein for delivery of a macrocyclic peptide to HeLa cells, wherein the macrocyclic peptide is admixed with the HeLa cells after the HeLa cells are subjected to mechanoporation.

HeLa cells were cultured in DMEM+GlutaMAX containing 10% FBS and 1% Pen/Strep in T225 flasks and lifted into single cell suspension using Trypsin EDTA 0.25%. Cells were passed through a 40 μm cell strainer then resuspended in Opti-MEM at 10M/mL.

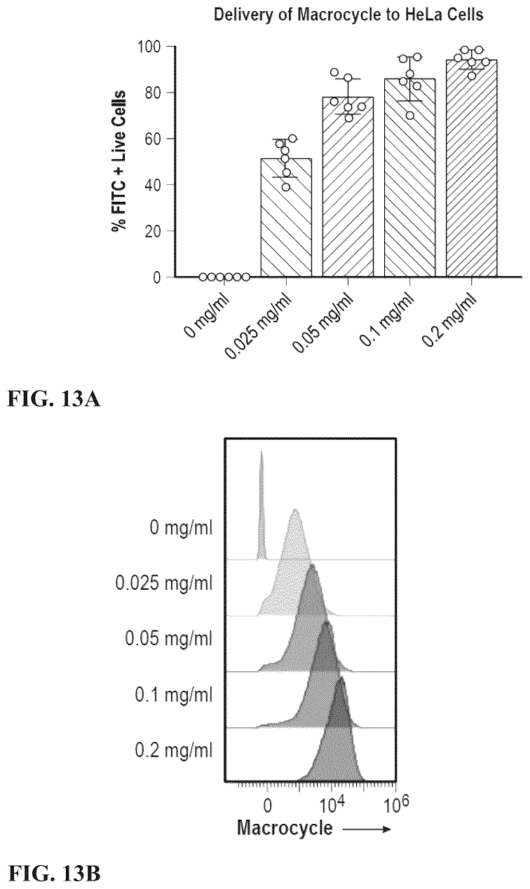

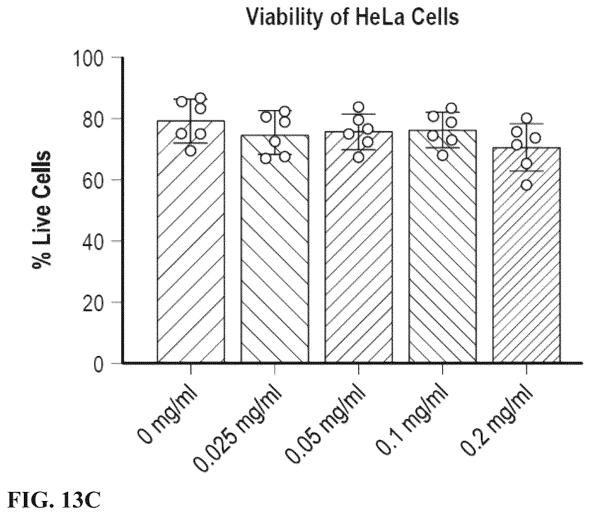

A 2× macrocyclic peptide solution containing 0.2 mg/ml of 3 kDa Cascade blue dextran and FITC-labeled macrocyclic peptide at 0, 0.05, 0.01, 0.2, or 0.4 mg/ml (0, 21, 42, 84, 168 μM) was prepared in Opti-MEM. 25 μL of the macrocyclic peptide solution was added to wells of a V-bottom 96 well plate. 150 μL aliquots of the cell suspension (1.5 million cells) were loaded into 10 and 11 μm pore size cartridges containing a filter and mechanoporated at 9 PSI. The cell suspension was collected in a 1.5 mL tube and 25 μL of cells were pipetted into wells of the 96-well plate containing cargo within 30 seconds of mechanoporation for a final cargo concentration of 0, 0.025, 0.05, 0.1, 0.2 mg/ml (˜0, 10.5, 21, 42, 84 μM). 200 μL aliquots of complete growth media (DMEM+GlutaMAX containing 10% FBS and 1% Pen/Strep) was added to the cell and macrocyclic peptide mixture approximately 30 seconds following addition of cells to the well. Delivery of dextran and the FITC-labeled macrocyclic peptide to HeLa cells were assessed by flow cytometry following delivery. Results demonstrate that introduction of the payload to a cell after subjecting to mechanoporation provided effective delivery and the processed cells maintained a high percentage of viability (FIG. 13A-13C).

1-114. (canceled)

115. A high-throughput system configured for distribution of mechanically perturbed cells, the high-throughput system comprising:
a cell reservoir configured to hold a liquid carrier comprising a plurality of cells;
a filter configured to be placed in fluidic communication with the cell reservoir, the filter comprising a plurality of pores therethrough for perturbing cell membranes of the plurality of cells;
a fluid controller configured to control the flow of the liquid carrier from the cell reservoir through the filter to produce the mechanically perturbed cells; and
a collection well configured to collect the liquid carrier comprising the mechanically perturbed cells from the filter and configured to accept a distributor comprising one or more dispensing tips for dispensing the liquid carrier comprising the mechanically perturbed cells into two or more samples.

116. The high-throughput system of claim 115, further comprising the distributor comprising the one or more dispensing tips for dispensing the liquid carrier comprising the mechanically perturbed cells into the two or more samples.

117. The high-throughput system of claim 115, wherein the plurality of pores in the filter is etched therein.

118. The high-throughput system of claim 115, wherein the plurality of pores comprises pores having a diameter between 2 μm and 20 μm.

119. The high-throughput system of claim 115, wherein the plurality of pores comprises uniformly sized pores.

120. The high-throughput system of claim 115, wherein the plurality of pores comprises pores having a diameter between 10% and 70% of a diameter of cells passed therethrough.

121. The high-throughput system of claim 115, wherein the filter has a thickness between 0.1 μm and 100 μm.

122. The high-throughput system of claim 115, wherein the filter comprises silicon, polymer, or variations thereof.

123. The high-throughput system of claim 115, further comprising tubing configured to deliver the plurality of cells from the cell reservoir to the filter.

124. The high-throughput system of claim 123, wherein the tubing has a length that is not more than about 200 times of the inner diameter of the tubing.

125. The high-throughput system of claim 123, wherein the tubing has an inner diameter (ID) of about 0.1 mm to about 10 mm.

126. The high-throughput system of claim 123, wherein the tubing has an inner diameter (ID) of at least about 0.2 mm.

127. The-high-throughput system of claim 115, further comprising a mesh cell strainer positioned proximal to the filter relative to the flow of the liquid carrier.

128. The high-throughput system of claim 115, wherein the high-throughput system comprises a single filter comprising the plurality of pores for perturbing cell membranes of the plurality of cells.

129. The high-throughput system of claim 115, wherein the filter is configured to process at least about 1 million cells.

130. The high-throughput system of claim 115, wherein the fluid controller further comprises a solenoid configured to create sample volumes of about 1 μL to about 500 μL.

131. The high-throughput system of claim 116, wherein the distributor comprises a single dispensing tip.

132. The high-throughput system of claim 115, wherein the cell reservoir is a syringe and the filter is placed in fluidic communication with the cell reservoir of the syringe via the outlet of the syringe.

133. A high-throughput system configured for distribution of mechanically perturbed cells, the high-throughput liquid handler comprising:
a cell reservoir configured to hold a liquid carrier comprising a plurality of cells;
a filter configured to be placed in fluidic communication with the cell reservoir, the filter comprising a plurality of pores therethrough for perturbing cell membranes of the plurality of cells;
a fluid controller configured to control the flow of the liquid carrier from the cell reservoir through the filter to produce the mechanically perturbed cells; and
a distributor comprising one or more dispensing tips for dispensing the liquid carrier comprising the mechanically perturbed cells into two or more samples.

134. The high-throughput system of claim 133, further comprising a collection well configured to collect the liquid carrier comprising the mechanically perturbed cells from the filter and configured to accept the distributor.

135. The high-throughput system of claim 134, wherein the cell reservoir is a syringe and the filter is placed in fluidic communication with the cell reservoir of the syringe via the outlet of the syringe.

136. A cartridge for use in the high-throughput system of claim 115, wherein the cartridge comprises the filter comprising the plurality of pores therethrough for perturbing cell membranes of the plurality of cells.

137. A kit comprising the cartridge of claim 136 and a collection well.

138. A cartridge for use in the high-throughput system of claim 133, wherein the cartridge comprises the filter comprising the plurality of pores therethrough for perturbing cell membranes of the plurality of cells.

139. A method of performing a cellular assay, the method comprising:
preparing a plurality of cell-containing samples using the high-throughput system of claim 115,
wherein the plurality of cell-containing samples comprises two or more samples separately having two or more populations of a cell comprising one or more different payloads delivered thereto; and
performing the cellular assay on plurality of cell-containing samples.

140. The method of claim 139, wherein the residency time, as measured as the time of a cell exiting the filter and having the payload delivered thereto, is about 2 minutes or less.

141. The method of claim 140, wherein the residency time is about 30 seconds or less.

142. A method of performing a cellular assay, the method comprising:
preparing a plurality of cell-containing samples using the high-throughput system of claim 133,
wherein the plurality of cell-containing samples comprises two or more samples separately having two or more populations of a cell comprising one or more different payloads delivered thereto; and
performing the cellular assay on plurality of cell-containing samples.

143. A method of performing a cellular assay, the method comprising
preparing a plurality of cell-containing samples comprising:
passing a plurality of cells through a filter comprising a plurality of pores therethrough for perturbing cell membranes of the plurality of cells;
distributing the mechanically perturbed cells to the plurality of cell-containing samples,
wherein the plurality of cell-containing samples comprises two or more samples separately having two or more populations of a cell comprising one or more different payloads delivered thereto; and
performing the cellular assay on plurality of cell-containing samples.

144. The method of claim 143, wherein the residency time, as measured as the time of a cell exiting the filter and having the payload delivered thereto, is about 2 minutes or less.
